(19) United States
(12) Patent Application Publication
(10) Pub. No.: US 2023/0158057 A1
(43) Pub. Date: May 25, 2023

(54) COMPOSITIONS AND METHODS FOR THE TREATMENT OF PAIN

(71) Applicant: Duke University, Durham, NC (US)

(72) Inventors: Christopher DONNELLY, Durham, NC (US); Ru-Rong JI, Durham, NC (US)

(73) Assignee: Duke University, Durham, NC (US)

(21) Appl. No.: 17/920,105

(22) PCT Filed: Apr. 21, 2021
(86) PCT No.: PCT/US2021/028384

§ 371 Date: Oct. 20, 2022

Related U.S. Application Data
(60) Provisional application No. 63/012,951, filed on Apr. 21, 2020.
(60) Provisional application No. 63/120,488, filed on Dec. 2, 2020.

(51) Int. Cl. A61K 31/7084; A61K 9/00; A61K 45/06; A61K 31/381; A61K 31/4184; A61P 29/00

(52) U.S. Cl. CPC A61K 31/7084; A61K 9/0019; A61K 45/06; A61K 31/381; A61K 31/4184; A61P 29/00

(57) ABSTRACT

The present disclosure methods and kits for use of stimulator of interferon genes (STING) agonist compounds, such as cyclic dinucleotides, amidobenzimidazoles, and benzothiophenes, in the treatment and prevention of pain, such as neuropathic pain, inflammatory pain, and cancer pain. Combination therapies and pharmaceutical formulations for treating of pain are also described.

### CROSS-REFERENCES TO RELATED APPLICATIONS

The present application claims priority to U.S. Provisional Pat. Appl. No. 63/012,951, filed on Apr. 21, 2020, and U.S. Provisional Pat. Appl. No. 63/120,488, filed on Dec. 2, 2020, which applications are incorporated herein by reference in their entirety.

### BACKGROUND OF THE INVENTION

Pain, including neuropathic pain, is a major health problem, affecting over 30% of Americans every year and costing more than $625 billion USD every year. Current treatments are only partially effective and cause significant side effects (e.g., addition by opioids). There is an urgent demand and need for effective and safe pain medicine.

### BRIEF SUMMARY OF THE INVENTION

This summary is provided to introduce a selection of concepts that are further described below in the Detailed Description. This summary is not intended to identify key or essential features of the claimed subject matter, nor is it intended to be used as an aid in limiting the scope of the claimed subject matter.

The present disclosure is based, in part, on the discovery by the inventors that both natural and synthetic agonists of the protein stimulator of interferon genes (STING) have analgesic effects of neuropathic pain.

Accordingly, one aspect of the present disclosure comprises a method of preventing a subject from developing pain and/or treating a subject suffering from pain comprising, consisting of, or consisting essentially of administering to the subject a therapeutically effective amount of a compound capable of modulating the activity of the stimulator of interferon genes (STING) receptor such that the pain is treated and/or prevented from developing in the subject.

In some embodiments, the compound comprises a STING agonist. In one embodiment, the STING agonist is selected from the group consisting of 3′3′-cGAMP, 2′3′-cGAMP, ADU-S100, MK-1415, MK-1454, and combinations thereof and pharmaceutical compositions thereof.

In some embodiments, the STING agonist is administered to the subject's dorsal root ganglia, skin, muscle, joint or cerebral spinal fluid (CSF). In some embodiments, the STING agonist is administered intrathecally to the cerebral spinal fluid.

In some embodiments, the method further comprises administering to the subject at least one additional therapeutic agent. In some embodiments, the at least one additional therapeutic agent is administered prior to the STING agonist. In some embodiments, the at least one additional therapeutic agent is administered concurrently with the STING agonist. In some embodiments, the at least one additional therapeutic agent is administered after the STING agonist.

In some embodiments, the at least one additional therapeutic agent is selected from the group consisting of PD-L1 and derivatives thereof, small molecular activators of PD-1, SHP-1 phosphatase activators, anti-inflammatory molecules, NSAIDS, steroids, opioids, local anesthetics, and combinations thereof.

Other aspects of the present disclosure provides a kit for the treatment of pain comprising, consisting of, or consisting essentially of a therapeutically effective amount of a STING modulator as provided herein, an apparatus for administering said STING modulator and instructions for use. In some embodiments, the kit further provides at least one additional therapeutic agent as provided herein and an apparatus for administering the at least one additional therapeutic to the subject (e.g., a tablet, a capsule, a syringe, a needle, a drip chamber, an inhaler, a nebulizer, a transdermal patch, an implant, or the like).

Another aspect of the present disclosure provides all that is described and illustrated herein.

### BRIEF DESCRIPTION OF THE DRAWINGS

FIG. 1A shows that STING agonists elevated mechanical thresholds for up to 48 h in naïve mice were administered vehicle or STING agonists via intrathecal (i.t.) injection for two successive days, followed by von Frey testing at the indicated timepoints.

FIG. 1B shows that STING agonists did not affect motor function in rotarod test in naïve mice were administered vehicle or STING agonists via intrathecal (i.t.) injection for two successive days, followed by von Frey testing at the indicated timepoints.

FIG. 1C shows that i.t. injection of the natural STING ligands 2′3′-cGAMP and 3′3′-cGAMP elevated mechanical thresholds in naïve mice.

FIG. 1D shows data from a syngeneic bone cancer pain model (BCP) established in test animals, followed by i.t. vehicle or STING agonist 10d post-inoculation. STING agonists suppressed BCP-induced mechanical allodynia.

FIG. 1E shows an experimental layout to test whether i.t. STING agonists can suppress ongoing pain in the BCP model using a conditioned place preference (CPP) assay (top panel). CPP was observed in STING agonist-paired mice compared to vehicle-paired mice (bottom panel).

FIG. 2A shows intrathecal administration of STING agonists increased IFN-α in DRG lysate 24 h following injection in WT, but not STINGgt/gt mice. The data indicate that STING inhibits nociception via type-I interferon signaling in nociceptors.

FIG. 2B shows that STING agonists elevated mechanical thresholds in Ifnar1fx/fx (WT) but not Ifnar1fx/fx; Nav1.8-Cre (cKO) mice.

FIG. 2C shows that IFN-α induced transient antinociception in WT mice which was abolished in Ifnar1-gKO/cKO mice.

FIG. 2D shows that IFN-β induced transient antinociception in WT mice which was abolished in Ifnar1-gKO/cKO mice.

FIG. 2E shows the quantification of current evoked action potentials in patch clamp recordings in dissociated DRG neurons from Ifnar1+/+ or Ifnar1−/− mice after acute perfusion with vehicle or rIFN-I. IFN-I inhibited action potential firing in DRG neurons from WT, but not gKO mice.

FIG. 3A shows that administration of ADU-S100 via i.t. catheter in non-human primates (Macaca mulatta) attenuated 2% menthol gel-induced cold allodynia in a dose-dependent manner. Statistical comparisons were conducted with two-way ANOVA with

Dunnett's post-hoc test (FIG. 3A, 3D), one-way ANOVA (FIG. 3B), two-tailed t-test (FIG. 3D), or one-sample t-test (vs. hypothetical value of 0).

FIG. 3B shows that administration of ADU-S100 via i.t. catheter in non-human primates (Macaca mulatta) increased IFN-β in cerebrospinal fluid (CSF) in NHPs treated with 3 nmol ADU-S100.

FIG. 3C shows quantification of action potentials in patch clamp recordings on DRG nociceptors from NHPs acutely treated with vehicle (top trace) or rIFN-I (bottom trace).

FIG. 3D shows quantification of rheobase in patch clamp recordings on DRG nociceptors from NHPs acutely treated with vehicle or rIFN-I. The patch claim data shows that rIFN-I perfusion inhibited NHP nociceptor excitability, as evidenced by reduced action potential firing and increased rheobase.

FIG. 3E shows representative recording following acute application of rIFN-I applied to a small-diameter (<55 μm) human DRG neuron (hDRG) neuron, with pipette attached for patch clamp recordings. Application of rIFN-I led to hyperpolarization of the membrane potential.

FIG. 3F shows the quantification of data in FIG. 3E.

FIG. 4A shows Sting1 (STING) mRNA expression in sensory neuron populations recently profiled and described by Zheng et al. (2019). Peptidergic nociceptive sensory neurons exhibit the highest expression of STING.

FIG. 4B shows the quantification of somal diameter in in situ hybridization of Sting1 in adult DRG sensory neurons using RNAscope, in conjunction with Niss1 staining to label all neurons Quantification of somal diameter in STING+ and STING-neurons indicated that STING-expressing neurons are primarily small-diameter sensory neurons.

FIG. 5A shows data collected from naïve mice that were administered vehicle or the STING agonist DMXAA via i.t. injection (arrows), followed by Von Frey testing to determine mechanical thresholds at 4 h following the 1st (day 1, D1) or 2nd (D2) injection. STING agonists induced a dose-dependent increase in paw withdrawal thresholds, which was further amplified by multiple injections. 10 μg (35 nmol) exhibited the largest effect, and therefore, was used throughout the rest of the study.

FIG. 5B shows data collected from naïve mice that were administered vehicle or ADU-S100, a STING agonist with cross-species activity, via i.t. injection (arrows) and tested as in panel a. 25 μg (35 nmol) exhibited the largest increase in paw withdrawal thresholds and this dose was used throughout the rest of the study.

FIG. 5C shows that systemic administration of DMXAA and ADU-S100 increased paw withdrawal threshold in naïve mice for up to 24 h.

FIG. 5D shows that the CIPN model, i.p. DMXAA and ADU-S100 suppressed mechanical allodynia for up to 48 h. Some toxicity was observed with systemic administration in the CIPN model, as 3 mice in the DMXAA group died 24 h after the 2nd injection. No mice died in the vehicle or ADU-S100 groups.

FIG. 5E shows that administration of DMXAA and ADU-S100 also suppressed cold allodynia (top panel) in the BCP model. These effects were not secondary to direct antitumor effects, as tumor burden was unaffected by STING agonist treatment (bottom panel).

FIG. 5F shows the effects of naloxone on morphine-, DMXAA-, and ADU-S100-induced antinociception. Naloxone (10 mg/kg, i.p.) reversed morphine (2 nmol i.t.)-induced antinociception (top left panel) but had no effect on the antinociceptive effects of DMXAA (35 nmol, i.t.) (top right panel) or ADU-S100 (35 nmol, i.t.) (bottom panel).

FIG. 5G shows that mechanical allodynia (top panel) and cold allodynia (bottom panel) were significantly reduced in DMXAA-treated mice at later timepoints (starting at D12). All data are expressed as the mean±s.e.m. * P<0.05; ** P<0.01; *** P<0.001, **** P<0.0001. Statistical comparisons were conducted with two-way ANOVA with Dunnett's (FIG. 5A-E), Bonferroni's (FIG. 5G), or Tukey's post-hoc test (FIG. 5F).

FIG. 6A shows that repeated pairing with i.t. morphine (2 nmol), but not DMXAA or ADU-S100 (35 -nmol), induces CPP in naïve mice.

FIG. 6B shows that a single pairing with i.t. DMXAA and ADU-S100 (35 nmol) induced comparable CPP as clonidine (35 nmol), a strong analgesic when administered via i.t. injection. All data are expressed as the mean±s.e.m. * P<0.05; ** P<0.01; *** P<0.001, **** P<0.0001. Statistical comparisons were conducted with one-way ANOVA with Fisher's LSD test. The data indicate that STING agonists induce conditioned place preference in mice with neuropathic pain but not in naïve mice.

FIG. 7A shows that the intrathecal administration of STING agonists increases IFN-α in serum 24 h following injection in WT, but not STINGgt/gt mice.

FIG. 7B shows that while basal IFN-β could be detected in DRG tissue from all genotypes, STINGgt/gt mice exhibited significantly lower IFN-β levels. c-d. ADU-S100 treatment of high density DRG neuron cultures from STING+/+ or STINGgt/gt mice. All data are expressed as the mean±s.e.m. * P<0.05; ** P<0.01; *** P<0.001, **** P<0.0001. Statistical comparisons were conducted with one-way ANOVA with Tukey's post-hoc test (FIG. 7A-B). The data indicate that STING agonists induce IFN-I production in sensory neurons in vitro and in vivo.

FIG. 8A shows that inhibition of endogenous IFN-I signaling via i.t. administration of an anti-IFN-α neutralizing antibody induced transient mechanical allodynia in naïve mice.

FIG. 8B shows that inhibition of endogenous IFN-I signaling via i.t. administration of an anti-IFN-β neutralizing antibody (vs. IgG control, 300 ng) induced transient mechanical allodynia in naïve mice. The data indicate that Type-I interferons regulate nociception in mice via Tyk2.

FIG. 8C shows that inhibition of the IFN-I signaling adapter Tyk2 with PF-06700841 (i.t., 1 μg) induced transient, dose-dependent mechanical allodynia in naïve mice.

FIG. 8D shows that i.t. injection of recombinant murine IFN-α increased paw withdrawal thresholds in naïve mice.

FIG. 8E shows that i.t. injection of recombinant murine IFN-β (produced in mammalian cells) increased paw withdrawal thresholds in naïve mice.

FIG. 8F shows that i.t. injection of recombinant universal IFN-I increased paw withdrawal thresholds in naïve mice. Notably, 100 U exhibited the greatest effects for each recombinant ligand in FIGS. 8C-8F. At higher concentrations some mice exhibited mechanical hypersensitivity.

FIG. 8G shows that pretreatment of naïve mice with the Tyk2 inhibitor PF-06700841 (i.t., 1 μg) abolished IFN-β-induced antinociception. All data are expressed as the mean±s.e.m. * P<0.05; ** P<0.01; *** P<0.001, **** P<0.0001. Statistical comparisons were conducted with two-way ANOVA with Bonferroni's (FIG. 8A-B) or Dunnett's post-hoc test (FIG. 8C-G).

FIG. 9A shows data from a study of peripheral and central actions of STING-mediated IFN-I signaling in DRG and spinal cord. L1-L5 DRGs were isolated from STINGfx/fx, STINGfx/fx; Nav1.8-Cre, and STINGgt/gt mice and incubated ex vivo with vehicle (left DRGs) or 30 μM ADU-S100 (right DRGs) for 2 h, followed by lysis and IFN-α and IFN-β ELISA. IFN-α levels in DRG lysate were increased by ex vivo incubation with ADU-S100 in WT mice.

FIG. 9B shows that IFN-β levels in DRG lysate were increased by ex vivo incubation with ADU-S100 in WT mice. ADU-S100 induced a significant elevation of IFN-β in STINGfx/fx; Nav1.8-Cre DRG lysate, but this increase was significantly lower than that seen in WT mice. All data are expressed as the mean±s.e.m. * P<0.05; ** P<0.01; *** P<0.001, **** P<0.0001. Statistical comparisons were conducted with one-way or two-way ANOVA with Tukey's test (9B-C).

FIG. 10A shows that i.t. poly(dA:dT) elevated mechanical thresholds in WT and RIG-I−/− mice for up to 24 h, but this effect was abolished in Ifnar1fx/fx; Nav1.8-Cre, STINGgt/gt, and cGAS−/− mice.

FIG. 10B shows that i.t. poly(I:C) elevated mechanical thresholds similarly in Ifnar1fx/fx (WT), STINGgt/gt, and cGAS−/− mice with a similar timecourse to poly(dA:dT), but these effects were abolished in Ifnar1fx/fx; Nav1.8-Cre and RIG-I−/− mice. e-g. Sodium currents were recorded from DRG nociceptors cultured from Ifnar1+/+ or Ifnar1−/− mice, perfused with vehicle or IFN-I for 2 minutes as indicated.

FIG. 10C shows the timecourse of Nav1.7-mediated currents. IFN-I perfusion reduced Nav1.7-mediated currents. All data are expressed as the mean±s.e.m. * P<0.05; ** P<0.01; *** P<0.001, **** P<0.0001. Statistical comparisons were conducted with two-way ANOVA with Dunnett's post-hoc test (FIG. 10A-B) or two-way ANOVA with Bonferroni's post-hoc test (FIG. 10C). The data indicate that dsDNA induces antinociception via the cGAS/STING/IFN-I pathway and proposed mechanism of STING/IFN-I regulation of nociception.

FIG. 11A shows Von Frey testing (left panel) to measure cancer-induced mechanical allodynia in mice treated with vehicle (left bars), ADU-S100 (20 mg/kg, i.p.; middle bars) or ZA (100 μg/kg, i.p.; right bars), n=7-8 mice per group. The right panel shows measurement of cancer-induced cold allodynia in mice with indicated treatment on day 7, 10 and 14 after LLC inoculation (n=7-8).

FIG. 11B shows spontaneous pain as determined by flinching behaviors (top left) or guarding behaviors (top right) over a 2 minute interval on d14 post inoculation (n=7-8). The bottom panel shows body weight measurements in mice with the indicated treatments (n=7-8). Vehicle: left bars; ADU-S100: middle bars; ZA: right bars. All data displayed represent the mean±SEM. *P<0.05, **P<0.01 and ***P<0.001, repeated-measures two-way ANOVA with Bonferroni's post-hoc test (11A); one-way ANOVA with Bonferroni's post-hoc test (11B).

FIG. 12A shows data from a radiographical analysis of bone destruction in mice administered vehicle (left bars), ADU-S100 (middle bars), or ZA (right bars) at the indicated timepoints after tumor inoculation.(n=7-8 mice/group). Data indicate the mean±SEM. *P<0.05, **P<0.01 and ***P<0.001, repeated-measures two-way ANOVA with Bonferroni's post-hoc test.

FIG. 12B shows of the quantification of tumor bearing femora with bone fractures after the indicated treatment harvested on d17 after LLC inoculation n=7-8 mice/group). Data indicate the mean±SEM. *P<0.05, **P<0.01 and ***P<0.001, repeated-measures two-way ANOVA with Fisher's exact test.

FIG. 13 shows quantification of TRAP staining to reveal osteoclast numbers in the tumor-bearing distal femora from mice treated with vehicle or DMXAA (2×20 mg/kg, i.p.) measured on d11 after LLC inoculation (n=5 mice/group). Data indicate the mean±SEM. *P<0.05, **P<0.01 and ***P<0.001, two-tailed Student's t-test.

### DETAILED DESCRIPTION OF THE INVENTION

The innate immune regulator STING is a critical sensor of self- and pathogen-derived DNA, leading to the induction of type-I interferons (IFN-I) and other cytokines which promote immune cell-mediated eradication of pathogens and neoplastic cells. STING has also emerged as a robust driver of antitumor immunity, leading STING activators and small molecule agonists to be developed as cancer immunotherapy adjuvants. Pain, transmitted by peripheral nociceptive sensory neurons (nociceptors), also aids in host defense by alerting organisms to the presence of potentially damaging stimuli, including pathogens and cancer cells. As described in detail below, it has now been discovered that STING is a critical regulator of nociception through IFN-I signaling in peripheral nociceptors. It is demonstrated, for example, that mice lacking STING or IFN-I signaling exhibit hypersensitivity to nociceptive stimuli and heightened nociceptor excitability. Conversely, intrathecal activation of STING has now been found to produce robust antinociception in mice and non-human primates (NHPs). It is believed that STING-mediated antinociception is governed by IFN-Is, which rapidly suppress excitability of mouse, NHP, and human nociceptors. These findings establish the STING/IFN-I signaling axis as a critical regulator of physiological nociception and a promising new target to combat chronic pain.

Agonists of the innate immune regulator stimulator of interferon genes (STING) have shown great efficacy in promoting antitumor immunity in preclinical models, leading to their exploration in cancer immunotherapy trials. Patients with advanced stage cancers frequently suffer from severe pain as a result of bone metastasis and bone destruction, for which there is no efficacious treatment. As described below, using multiple mouse models of metastatic bone cancer, STING agonists have now been found to confer remarkable protection against cancer pain, bone destruction, and local tumor burden. Repeated systemic administration of STING agonists robustly attenuated bone cancer-induced pain symptoms and improved locomotor function. Interestingly, STING agonists provided acute pain relief through direct neuronal modulation, as ex vivo incubation of STING agonists reduced excitability of pain-sensing nociceptive neurons from tumor-bearing mice. In addition, STING agonists protected local bone destruction and reduced local tumor burden through modulation of osteoclast and immune cell function in the tumor microenvironment, providing long-term cancer pain relief. Finally, these in vivo effects were dependent on host-intrinsic STING and Ifnar1. Overall, STING activation provides unique advantages in controlling metastatic bone cancer pain through distinct and synergistic actions on nociceptors, immune cells, and osteoclasts.

### I. DEFINITIONS

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the term “pain” refers to the basic bodily sensation induced by a noxious stimulus, received by naked nerve endings, characterized by physical discomfort (e.g., pricking, throbbing, aching, etc.) and typically leading to an evasive action by the individual. The pain may be chronic or acute. As used herein, the term pain also includes, but is not limited, neuropathic pain, inflammatory pain, and cancer pain.

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule. For example, the compositions described herein may act as antagonists and modulate (i.e., inhibit/downregulate) STING activity. In some embodiments, the compositions described herein may act as agonists and modulate (i.e., increase/upregulate) STING activity.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, tissue, mucus and tears. In one embodiment, the biological sample is a blood sample (such as a plasma sample). A biological sample may be obtained directly from a subject (e.g., by blood or tissue sampling) or from a third party (e.g., received from an intermediary, such as a healthcare provider or lab technician).

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e., living organism, such as a patient).

Unless otherwise defined, all technical terms used herein have the same meaning as commonly understood by one of ordinary skill in the art to which this disclosure belongs.

### II. METHODS FOR TREATMENT OF PAIN

Pain, transmitted by peripheral nociceptive sensory neurons (nociceptors), also aids in host defense by alerting organisms to the presence of potentially damaging stimuli, including pathogens and cancer cells. Here, the inventors provide a link between these two ancient defense systems, demonstrating that STING is a critical regulator of nociception in physiological and pathological states through IFN-I signaling in peripheral nociceptors.

Accordingly, one aspect of the present disclosure comprises a method of preventing a subject from developing pain and/or treating a subject suffering from pain comprising, consisting of, or consisting essentially of administering to the subject a therapeutically effective amount of a compound capable of modulating the activity of the stimulator of interferon genes (STING) receptor such that the pain is treated and/or prevented from developing in the subject.

In some embodiments, the methods are employed for the treatment of neuropathic pain, inflammatory pain, cancer pain, or any combination thereof. The terms “neuropathic pain” or “neurogenic pain” can be used interchangeably and refer to pain that arises from direct stimulation of nervous tissue itself, central or peripheral and can persist in the absence of stimulus. The sensations that characterize neuropathic pain vary and are often multiple and include burning, gnawing, aching, and shooting. (See, e.g., Rooper and Brown, (2005) Adams and Victor's Principles of Neurology, 8^{th} ed., NY McGraw-Hill). These damaged nerve fibers send incorrect signals to other pain centers. The impact of nerve fiber injury includes a change in nerve function both at the site of injury and areas around the injury, as well as in the central nervous system. Chronic neuropathic pain often seems to have no obvious cause, however, some common causes may include, but are not limited to, alcoholism, amputation, back, leg and hip problems, chemotherapy, diabetes, facial nerve problems (e.g., trigeminal neuralgia), HIV infection or AIDS, multiple sclerosis, shingles, spine surgery, spinal cord injury, traumatic brain injury, and stroke. For example, one example of neuropathic pain is phantom limb syndrome, which occurs when an arm or leg has been removed because of illness or injury, but the brain still gets pain messages from the nerves that originally carried impulses from the missing limb. Also included within the definition of pain include inflammatory pain (pain as the result of the inflammation, e.g., inflammatory hyperalgesia such as arthritis) and “other pain” (e.g., cancer pain, muscle pain, and headache). Mechanical allodynia or tactile allodynia, pain induced by normally innocuous mechanical stimulation, is a common feature of chronic pain. Chronic pain due to arthritis and cancer are also serious problems in pets and other companion animals.

In some embodiments, the pain comprises an inflammatory pain. In some embodiments, the pain comprises neuropathic pain. In some embodiments, the pain comprises cancer pain, headaches, or a combination thereof. In some embodiments, the pain comprises mechanical allodynia or cold allodynia. For example, a subject afflicted with neuropathic pain or cancer pain may experience increased sensitivity to relatively innocuous stimuli such as light touch or cool or cold temperatures. The pain may be characterized by spontaneous occurrence (e.g., at irregular intervals, including intervals with little predictability), transient occurrence (e.g., for periods of time ranging from a few minutes to a few hours), and/or ongoing occurrence (e.g., for hours, days, or longer). The frequency and/or duration may vary from one period of time to another. In some embodiments, the pain is not cancer pain. In some embodiments, the subject to whom the STING agonist is administered does not have cancer.

A. STING Agonists

Any suitable STING agonist may be administered for treatment of pain according to the present disclosure. Such STING agonists include, but are not limited to, those described by Aval et al (J. Clin. Med. 2020, 9, 3323), which is incorporated herein by reference in its entirety. In some embodiments, the STING agonist is a cyclic dinucleotide. The term “cyclic dinucleotide” refers to a compound than contains two nucleosides covalently bonded to each other via phosphoester linkages between two ribose hydroxyl groups of the first nucleoside and two ribose hydroxyl groups of the second nucleoside. Examples of cyclic dinucleotides useful in the methods of the present disclosure include, but are not limited to, 2′3′ cyclic guanosine monophosphate—adenosine monophosphate (2′3′-cGAMP; CAS Registry No. 1441190-66-4); 3′3′-cGAMP (CAS Registry No. 849214-04-6), cyclic diAMP (cdA; CAS Registry No. 54447-84-6), cyclic diGMP (cdG; CAS Registry No. 61093-23-0), and pharmaceutically acceptable salts thereof. In some embodiments, the cyclic dinucleotide is a cyclic dinucleotide thiophosphate. The cyclic dinucleotide thiophosphate may contain one or two phosphorothioate moieties, in which one of the nonbridging oxygen atoms in the phosphoester linkage is replaced by a sulfur. Examples of cyclic dinucleotide thiophosphates include, but are not limited to, (2′-5′)-[P(R)]-5′-O-[(R)-hydroxymercaptophosphinyl]-P-thioadenylyl-adenosine cyclic dinucleotide, also referred to as ADU-S100, pharmaceutically acceptable salts, and other cyclic dinucleotide thiophosphates described in WO 2014/189806, which is incorporated herein by reference in its entirety. Further examples of cyclic dinucleotides include those described in U.S. Pat. Nos. 9,724,408; 8,367,716; 7,709,458; 7,592,326; WO 2018/118665; WO 2018/172206; WO 2018/198076; and WO 2020/016782; which references are incorporated herein by reference in their entirety. In some embodiments, the cyclic dinucleotide is 3′3′-cGAMP, 2′3′-cGAMP, ADU-S100 (CAS No. 1638750-95-4), or a combination thereof.

In some embodiments, the STING agonist is an oxoxanthenyl carboxylic acid (e.g., 2-(5,6-dimethyl-9-oxo-9H-xanthen-4-yl)acetic acid), an oxochromenyl carboxylic acid (e.g., 2-(4-oxo-2-phenyl-4H-chromen-8-yl)acetic acid) or an oxoacridinyl carboxylic acid (e.g., 2-(9-oxoacridin-10(9H)-yl)acetic acid).

In some embodiments, the STING agonist is an amidobenzimidazole such as a dimerized amidobenzimidazole (e.g., as described in WO 2019/069270, which is incorporated herein by reference in its entirety. A non-limiting example of such STING agonists is (E)-1-((E)-4-((E)-5-carbamoyl-2-((1-ethyl-3-methyl-1H-pyrazole-5-carbonyl)imino)-7-(3-morpholinopropoxy)-2,3-dihydro-1H-benzo[d]imidazol-1-yl)but-2-en-1-yl)-2-((1-ethyl-3-methyl-1H-pyrazole-5-carbonyl)imino)-7-methoxy-2,3-dihydro-1H-benzo[d]imidazole-5-carboxamide (see also, Ramanjulu et al. Nature 2018, 564: 439-443).

In some embodiments, the STING agonist is a 3-oxo-3,4-dihydro-2H-benzo[b-1,4]-thiazine-6-carboxylate, optionally containing further substituents at the 2-position (e.g., as described in WO 2018/234805, which is incorporated herein by reference in its entirety).

In some embodiments, the STING agonist is a 3,3-dimethyl-2-oxoindoline-6-carboxylate, optionally containing further substituents at the 1-position (e.g., as described in WO 2018/234807, which is incorporated herein by reference in its entirety).

In some embodiments, the STING agonist is a 3-alkyl-3,4-dihydro-1H-benzo[c][1,2,5]thiadiazine-7-carboxylate 2,2,-dioxide, optionally containing further substituents at the 1-position (e.g., as described in WO 2018/234808, which is incorporated herein by reference in its entirety.

In some embodiments, the STING agonist is a benzo[b]thiophene, an aza-benzothiophene, or a benzothiophene (e.g., as described in WO 2019/027858, WO 2019/195063, and WO 2019/195124, which are incorporated herein by reference in their entirety). A non-limiting example of one such STING agonist is 4-(5,6-dimethoxybenzo[b]thiophen-2-yl)-4-oxobutanoic acid, also referred to a MSA-2.

In some embodiments, the STING agonist is a 3-carboxamido-thiophene-2-carboxylic acid, a 3-carboxamido-picolinic acid, a 3-carboxamidobenzoic acid, or a 2-substituted 4H-benzo[d][1,3]oxazin-4-one (e.g., as described in WO 2021/035258, which is incorporated herein by reference in its entirety. A non-limiting example of one such STING agonist is 2-(6-(1H-imidazol-1-yl)pyridazine-3-carboxamido)-4,5-difluorobenzoic acid, also referred to as SR-717.

In some embodiments, the STING agonist is selected from the group consisting of 3′3′-cGAMP, 2′3′-cGAMP, ADU-S100 (CAS No. 1638750-95-4), MK-1454, and combinations thereof, which may be administered, for example, in a pharmaceutical composition such as one described herein.

B. Dosing and Administration

The STING modulating compounds, and pharmaceutical compositions thereof, may be administered to a subject by any technique known in the art, including local or systemic delivery. Routes of administration include, but are not limited to, subcutaneous, intravenous, intrathecal, intramuscular, epidural injection or implantation, or intranasal administration. In some embodiments, the compound is administered intrathecally (e.g., an administration into the spinal canal, or into the subarachnoid space, or into space under the arachnoid membrane of the brain) or intravenously (IV). In some embodiments, the compound is administered to the subject's skin, muscle, joint, cerebral spinal fluid (CSF) or dorsal root ganglia. In some embodiments, the subject is a human. In some embodiments, the STING agonist is administered intrathecally to the cerebral spinal fluid.

The STING modulating compounds, and pharmaceutical compositions thereof, may be administered in a single dose or in multiple doses (e.g., two, three, or more single doses per treatment) over a time period (e.g., hours or days). The STING agonist(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of STING agonist(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular STING agonist(s) the conversation rate and efficiency into active drug compound under the selected route of administration, etc.

Determination of an effective dosage of STING agonist(s) for a particular use and mode of administration is well within the capabilities of those skilled in the art. Effective dosages may be estimated initially from in vitro activity and metabolism assays. For example, an initial dosage of STING agonist for use in animals may be formulated to achieve a circulating blood or serum concentration that is at or above an IC50 of the particular STING agonist as measured in an in vitro assay. The dosage can be calculated to achieve such circulating blood or serum concentrations taking into account the bioavailability of the particular STING agonist via the desired route of administration. Initial dosages of compound can also be estimated from in vivo data, such as animal models. For example, an average mouse weighs 0.025 kg. Administering 0.025, 0.05, 0.1 and 0.2 mg of a cyclic dinucleotide per day may therefore correspond to a dose range of 1, 2, 4, and 8 mg/kg/day. If an average human adult is assumed to have a weight of 70 kg, the corresponding human dosage would be 70, 140, 280, and 560 mg of the cyclic nucleotide per day. Dosages for other active agents may be determined in similar fashion. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the STING agonist or other active compound, the bioavailability of the STING agonist or other active compound, its metabolism kinetics and other pharmacokinetic properties, the mode of administration and various other factors, discussed above. The dose of the STING agonist can be, for example, about 0.01-750 mg/kg, or about 0.01-500 mg/kg, or about 0.01-250 mg/kg, or about 0.01-100 mg/kg, or about 0.1-50 mg/kg, or about 1-25 mg/kg, or about 1-10 mg/kg, or about 5-10 mg/kg, or about 1-5 mg/kg. The dose of the STING agonist can be about 0.01, 0.05, 0.1, 0.5, 1, 5, 10, 15, 20, 25, 30, 35, 40, 45, 50, 55, 60, 65, 70, 75, 80, 85, 90, 95, or 100 mg/kg.

Dosage amount and interval may be adjusted individually to provide plasma levels of the STING agonist(s) and/or active metabolite STING agonist(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of STING agonist(s) and/or active metabolites thereof may not be related to plasma concentration. Skilled artisans will be able to optimize effective dosages without undue experimentation.

The compounds may be present in a therapeutically effective concentration. In certain embodiments, the concentration of said compound is about 0.1 nmol/L to about 1000 nmol/L at the time of administration; e.g., about 0.1 nmol/L to about 500 nmol/L, or about 0.1 nmol/L to about 250 nmol/L, or about 0.1 nmol/L to about 100 nmol/L, or about 0.1 nmol/L to about 50 nmol/L, or about 0.1 nmol/L to about 10 nmol/L, or about 0.1 nmol/L to about 1 nmol/L, or about 1 nmol/L to about 500 nmol/L, or about 1 nmol/L to about 250 nmol/L, or about 1 nmol/L to about 100 nmol/L, or about 1 nmol/L to about 50 nmol/L, or about 1 nmol/L to about 10 nmol/L, or about 10 nmol/L to about 500 nmol/L, or about 10 nmol/L to about 250 nmol/L, or about 10 nmol/L to about 100 nmol/L, or about 10 nmol/L to about 50 nmol/L, or about 100 nmol/L to about 500 nmol/L, or about 100 nmol/L to about 250 nmol/L. One of skill in the art will recognize that suitable volume of the dose may be selected based on the desired route of administration.

C. Combination Therapy

STING agonists as described herein may be administered in combination with additional therapeutic agents including, but not limited to, analgesics useful for treating pain. The STING agonists may be administered in the form of compounds per se, or as pharmaceutical compositions comprising one or more pharmaceutically acceptable excipients. Accordingly, methods in some embodiments further comprise administering to the subject at least one additional therapeutic agent. In general, additional therapeutic agents may be formulated and dosed as described herein with respect to a STING agonist such as a cyclic dinucleotide, an amidobenzimidazole, or a benzothiophene. In some embodiments, the at least one additional therapeutic agent is administered prior to the STING agonist. In some embodiments, the at least one additional therapeutic agent is administered concurrently with the STING agonist. In some embodiments, the at least one additional therapeutic agent is administered after the STING agonist. In some embodiments, the at least one additional therapeutic agent comprises a pain reliever or a therapeutic agent that can impart an analgesic effect on a subject.

In some embodiments, the additional therapeutic agent is a steroid (e.g., dexamethasone, cortisol, cortisone, hydrocortisone, prednisone, prednisolone, methylprednisolone, betamethasone, triamcinolone, beclometasone, fludrocortisone acetate, deoxycorticosterone acetate, aldosterone, or the like).

In some embodiments, the additional therapeutic agent is a nonsteroidal anti-inflammatory drug (NSAID; e.g., ibuprofen, naproxen, diclofenac, celecoxib, mefenamic acid, etoricoxib, indomethacin, high-dose aspirin, or the like). In some embodiments, the additional therapeutic agent is an opioid analgesic (e.g., codeine, dextropropyoxyphene, diamorphine, dihydrocodeine, meptazinol, methadone, morphine, nalbuphine, pentazocine, or the like). Analgesics that may also be used in the methods include, but are not limited to, aloxiprin, auranofin, azapropazone, benorylate, diflunisal, etodolac, fenbufen, fenoprofen calcium, ketoprofen, meclofenamic acid, nabumetone, oxyphenbutazone, phenylbutazone, piroxicam, and sulindac. In some embodiments, the additional therapeutic agent is a local anesthetic (e.g., articaine, benzocaine, bupivacaine, lidocaine, mepivacaine, prilocaine, or the like.)

In some embodiments, the additional therapeutic agent is cell death 1 ligand 1 (PD-L1; e.g., human PD-L1, Uniprot Accession No. Q9NZQ7) or a derivative thereof (e.g., a truncated PD-L1 polypeptide or a PD-L1 fusion protein) or an activator of PD-1 (e.g., a small molecule PD-1 activator). In some embodiments, the additional therapeutic is a SHP-1 phosphatase activator (e.g., a bisphenyl urea such as 1-(4-chloro-3-(trifluoromethyl)phenyl)-3-(3-(4-cyanophenoxy)phenyl)urea or those described in U.S. Pat. No. 10,745,346). The SHP-1 phosphatase inhibitor can, for example, increase potassium channel activation and/or decrease TPRV1 channel activation.

In some embodiments, the additional therapeutic agent is selected from programmed cell death 1 ligand 1 and derivatives thereof, a small molecular activator of PD-1, a SHP-1 phosphatase activator, an anti-inflammatory molecule (e.g., an NSAID), a steroid, an opioid, a local anesthetic, or a combination thereof.

In some embodiments, STING agonists may be administered in conjunction with one or more anti-cancer agents. Examples of anti-cancer agents include, but are not limited to, chemotherapeutic agents (e.g., carboplatin, paclitaxel, pemetrexed, or the like), tyrosine kinase inhibitors (e.g., erlotinib, crizotinib, osimertinib, or the like), and immunotherapeutic agents (e.g., pembrolizumab, nivolumab, durvalumab, atezolizumab, or the like). The STING agonists may also be administered in conjunction with radiotherapy, e.g., external beam radiation; intensity modulated radiation therapy (IMRT); brachytherapy (internal or implant radiation therapy); stereotactic body radiation therapy (SBRT)/stereotactic ablative radiotherapy (SABR); stereotactic radiosurgery (SRS); or a combination of such techniques.

D. Pharmaceutical Formulations

When used to treat or prevent such disorder, diseases, and or conditions associated with nociception receptor activity (e.g., pain), the compositions described herein may be administered singly, as mixtures of one or more compositions, or in a mixture or combination with other additional therapeutic agents useful for treating such diseases, disorders, and/or conditions and/or the symptoms associated with such diseases, disorders, and/or conditions (e.g., pain) as provided herein.

STING agonists as described herein may be formulated as pharmaceutical compositions containing an appropriate carrier, excipient or diluent. The exact nature of the carrier, excipient or diluent will depend upon the desired use for the composition and may range from being suitable or acceptable for veterinary uses to being suitable or acceptable for human use.

Pharmaceutical compositions comprising the STING agonist(s) may be manufactured by processes including or more mixing, dissolving, granulating, dragee-making, levigating, emulsifying, encapsulating, entrapping, and/or lyophilizing steps. The compositions may be formulated in conventional manner using one or more physiologically acceptable carriers, diluents, excipients or auxiliaries which facilitate processing of the STING agonists into preparations which can be used pharmaceutically.

The STING agonists may be formulated in the pharmaceutical composition per se, or in the form of hydrates, solvates, N-oxides, or pharmaceutically acceptable salts. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the STING agonist(s) may be formulated as solutions, gels, ointments, creams, suspensions, etc. as are well-known in the art. Systemic formulations include those designed for administration by injection, e.g., subcutaneous, intravenous, intramuscular, intrathecal, peri-neural, or intraperitoneal injection, as well as those designed for transdermal, transmucosal oral or pulmonary administration. In some embodiments, the STING agonist is administered to a cancer patient via intra-tumoral injection.

Useful injectable preparations include sterile suspensions, solutions or emulsions of the STING agonist(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the STING agonist(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats);

emulsifying agents (e.g., lecithin or acacia); non-aqueous vehicles (e.g., almond oil, oily esters, ethyl alcohol, CREMOPHORE™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the STING agonist(s) may be formulated as solutions (for retention enemas) suppositories or ointments containing conventional suppository bases such as cocoa butter or other glycerides.

For nasal administration or administration by inhalation or insufflation, the STING agonist(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the STING agonist(s) may be formulated as a solution, emulsion, suspension, etc. suitable for administration to the eye. A variety of vehicles suitable for administering compounds to the eye are known in the art.

For prolonged delivery, the STING agonist(s) can be formulated as a depot preparation for administration by implantation or intramuscular injection. The STING agonist(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the STING agonist(s) for percutaneous absorption may be used. To this end, permeation enhancers may be used to facilitate transdermal penetration of the STING agonist(s).

Alternatively, other pharmaceutical delivery systems may be employed. Liposomes and emulsions are well-known examples of delivery vehicles that may be used to deliver STING agonist(s). Certain organic solvents such as dimethyl sulfoxide (DMSO) may also be employed, although usually at the cost of greater toxicity.

In some embodiments, a STING agonist such as a cyclic dinucleotide can be delivered in a lipid-containing composition, optionally containing one or more excipients for increasing stability; permitting the sustained or delayed release (e.g., from a depot formulation); altering the biodistribution (e.g., target to specific tissues or cell types); and/or altering the release profile of the STING agonist in vivo. Such excipients may include solvents, dispersion media, diluents, dispersion or suspension aids, surface active agents, isotonic agents, thickening or emulsifying agents, preservatives. Such compositions include, without limitation, lipidoids, liposomes, lipid nanoparticles, lipoplexes, and combinations thereof.

In some embodiments, pharmaceutical compositions for STING agonist delivery may include liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see e.g., U.S. Pat. No. 8,283,333, which is incorporated herein by reference in its entirety). Such liposomes may contain 3, 4, or more lipid components. As an example a liposome can contain, but is not limited to, 50-60% cholesterol, 15-25% disteroylphosphatidyl choline (DSPC), 5-15% PEG-S-DSG, and 10-20% 1,2-dioleyloxy-N,N-dimethylaminopropane (DODMA). Cationic lipids such as 1,2-distearloxy-N,N-dimethylaminopropane (DSDMA), DODMA, DLin-DMA, or 1,2-dilinolenyloxy-3-dimethylaminopropane (DLenDMA), may also be included in the liposomes.

In some embodiments, a STING agonist may be formulated in a cationic oil-in-water emulsion where the emulsion particle comprises an oil core and a cationic lipid. Alternatively, the STING agonist may be formulated in a water-in-oil emulsion comprising a continuous hydrophobic phase in which the hydrophilic phase is dispersed. (see, e.g., WO 2012/006380, which is incorporated by reference in its entirety).

In some embodiments, the compositions include a STING agonist and a poloxamer as described, for example, in U.S. 2010/0004313, which is incorporated by reference in its entirety. Poloxamer formulations and other polymer formulations may be stabilized by contacting the formulation, which may include a cationic carrier, with a cationic lipopolymer which may be covalently linked to cholesterol and polyethylene glycol groups. The cationic carrier may include, but is not limited to, polyethylenimine, poly(trimethylenimine), poly (tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, chitosan, cationic lipids, and combinations thereof.

The pharmaceutical compositions may, if desired, be presented in a pack or dispenser device which may contain one or more unit dosage forms containing the STING agonist(s). The pack may, for example, comprise metal or plastic foil, such as a blister pack. The pack or dispenser device may be accompanied by instructions for administration.

Other aspects of the present disclosure provides a kit for the treatment of pain comprising, consisting of, or consisting essentially of a therapeutically effective amount of a STING modulator as provided herein, an apparatus for administering said STING modulator, and instructions for use. In some embodiments, the kit further provides at least one additional therapeutic agent as provided herein and an apparatus for administering the at least one additional therapeutic to the subject.

### III. EXAMPLES

The following Examples are provided by way of illustration and not by way of limitation.

### Example 1. Materials and Methods for Study of STING Activity in Nociception

Reagents: This study utilized the following reagents and concentrations: DMXAA (various concentrations, as indicated; Cayman Chemical, 14617), ADU-S100 (various concentrations, as indicated; Chemietek, CT-ADUS100), 2′3′-cGAMP (10 μg, i.t.; Invivogen, tlr1-nacga23-02), 3′3′-cGAMP (10 μg, i.t.; Invivogen, tlr-nacga), poly(dA:dT)/LyoVec (1 μg, i.t.; Invivogen, tlr1-patc), poly(I:C)/LyoVec (1 μg, i.t.; high molecular weight; Invivogen, tlr1-piclv), clonidine hydrochloride (35 nmol, i.t.; Millipore Sigma, C7897), naloxone hydrochloride dihydrate (10 mg/kg, i.p.; Millipore Sigma, N7758), morphine sulfate (2 nmol, i.t.; WEST-WARD Pharmaceuticals), recombinant mouse IFN-α A (various concentrations, as indicated; PBL Assay Science, 12100-1), recombinant mouse IFN-β (various concentrations, as indicated; PBL Assay Science, 12410-1), recombinant universal type-I interferon (various concentrations, as indicated; PBL Assay Science, 11200-1), H-151 (10 nmol, i.t.; Cayman Chemical, #25857), C-176 (10 nmol, i.t.; Cayman Chemical, #25859), PF-06700841 tosylate salt (various concentrations, as indicated; Millipore Sigma, PZ0345-5MG), U0126 (1 μg, i.t.; Millipore Sigma, 19-147), LY294002 (1 μg; Millipore Sigma, L9908), anti-mouse IFN-α neutralizing antibody (300 ng/mouse, i.t.; PBL Assay Science, 32100-1), anti-mouse IFN-β neutralizing antibody (300 ng/mouse, i.t.; PBL Assay Science, 32400-1), rabbit polyclonal IgG control (300 ng/mouse, i.t.; Biolegend, CTL-4112), paclitaxel (Millipore Sigma, T7191), and resiniferatoxin (Millipore Sigma, R8756).

Genetic mouse models: All mouse procedures were approved by the Duke University Institutional Animal Care and Use Committee (IACUC) and complied with relevant ethical guidelines. Mice were housed in an animal facility approved by the Association for Assessment and Accreditation of Laboratory Animal Care (AAALAC) under a 12 h light/dark cycle with food and water available ad libitum. All commercially-available genetic mouse models were obtained from Jackson Labs and maintained on a C57BL/6J background, including: Ifnb1YFP reporter mice (strain #010818) 1, STING “goldenticket” knockout mice 2 (STINGgt/gt; strain #017537), STING floxed/conditional knockout mice 3 (STINGfx/fx; strain #031670), Ifnar1 global knockout mice (Ifnar1−/−; strain #028288), Ifnar1 floxed/conditional knockout mice 4 (Ifnar1fx/fx; strain #028256), cGAS knockout mice 5 (cGAS−/−; strain #026554), and RIG-I knockout mice (RIG-I−/−; strain #46070-JAX). Nav1.8-Cre mice 6, also maintained on a C57BL/6J background, were a gift from Rohini Kuner (University of Heidelberg). NOD.CB17-Prkdcscid mice were also obtained from Jackson Labs (strain #001303) and maintained in a NOD/ShiLtSz genetic background. Unless otherwise noted, all experiments were conducted in adult (8-12-week-old) mice. Animals were randomly assigned to each experimental group. Both males and females were included in each group in a sex-matched manner. The data from both sexes were combined and used relatively equally throughout this study, as no sex differences were observed.

Mouse pain models: The chemotherapy-induced peripheral neuropathy (CIPN) model of chronic neuropathic pain was established as we have done previously, using 8-10-week-old CD-1 (strain #022, Charles River) mice injected with paclitaxel (2 mg/kg i.p.) every other day, with 4 injections total. The chronic constriction injury (CCI) model of neuropathic pain was also produced using 8-10-week-old CD-1 under isoflurane anesthesia. Briefly, the right sciatic nerve was exposed above the hip and two ligatures (7-0 Prolene) were placed around the nerve 1 mm apart located proximal to the trifurcation. Ligatures were loosely tied until a subtle flick of the ipsilateral hind limb was observed. CD-1 mice were also used to establish the SNI model, which was generated by tightly ligating the tibial and common peroneal nerves followed by transection and removal of a ˜3 mm portion of the nerve. The sural nerve was left intact and contact or stretching of this nerve was avoided . To establish the syngeneic bone cancer pain (BCP) model, 8-10-week-old C57BL/6J mice (strain #000664, Jackson Labs) were utilized, as the Lewis lung carcinoma cell line LLC1 (LL/2; ATCC CRL-1642) was originally generated in C57BL/6J mice. Mice were anesthetized with isoflurane and the left leg was shaved, disinfected with 10% povidone-iodine, and a 1 cm superficial incision was made to expose the patellar ligament. A 25-gauge needle was inserted at the intercondylar notch of the left femur into the femoral cavity, followed by needle replacement with a 10 μl Hamilton syringe microinjector containing a 2 μl suspension of LLC cells (2×105 cells) followed by a 2 μl gelatin sponge solution to enable closure of the injection site. All mice were housed and monitored in accordance with AAALAC standards and Duke IACUC guidelines that pertained to each model.

Reflexive-based sensory testing in mice: All behavioral testing in mice was performed in a specialized humidity- and light-controlled mouse behavior facility maintained at 21-24° C., with testing conducted between the hours of 8:00-16:00. Mice were habituated to the testing environment for at least 2 days prior to baseline testing. All tests were performed by an experimenter who was blinded to experimental conditions, including genotypes and drug treatment. For multi-day experiments using STING agonists, mice were tested in the same room at approximately the same time each day. To measure mechanical sensitivity, mice were confined to individual chambers with non-transparent borders on an elevated mesh rack, preventing mice from visualizing or interacting with one another during the testing period. Mouse hindpaws were stimulated with a series of von Frey filaments with logarithmically increasing stiffness (0.02-2.56 g, Stoelting), which was applied perpendicularly to the central plantar surface. We determined the 50% paw withdrawal threshold using the up-down method. We also assayed mechanical allodynia by determining paw withdrawal frequency to repeated stimulation (10 times, with ˜1-2 minutes between each stimulation) using a subthreshold 0.16 g von Frey filament, which ordinarily does not elicit reflexive withdrawal. To test cold sensitivity, mice were again placed on an elevated metal mesh floor in isolated chambers, and a ˜20 μl acetone drop was applied to the central plantar surface of the mouse hindpaw using a pipette. The duration of time that animals exhibited nociceptive behaviors (paw lifting, licking, flicking) was recorded using a stopwatch immediately following acetone application.

Naloxone reversal and repeated STING agonist administration: To test whether the opioid receptor antagonist naloxone could reverse morphine or STING agonist-induced antinociception, naloxone was administered (10 mg/kg, i.p.) at the following timepoints after drug delivery: morphine (2 nmol, i.t., 30 minutes), DMXAA (35 nmol, i.t., 4 h after administration), ADU-S100 (35 nmol, i.t., 4 h after administration). Following naloxone delivery, mice were tested within 15 minutes. For the experiments testing whether mice developed tolerance to the effects of STING agonists, vehicle or DMXAA (35 nmol) were administered via i.t. injection. Naïve mice received five single injections at D0, D3, D6, D9, and D12, with testing each day 4 h after injection. SNI mice received injections on two consecutive days beginning on D7 (D7 and D8) and every 48 h afterwards following the conclusion of sensory testing (D10, D12, D14, D16, and D18). Spinal cords (L4-L5) were collected for analysis of glial cell activation at D21 following SNI.

Motor, locomotor, and sensorimotor behaviors: Rotarod testing was performed to assess motor coordination, as we have done previously. Prior to testing, mice were placed in the behavioral room for 30 minutes. We used an accelerating protocol (4-45 RPM over 300 seconds). Each mouse was tested in 3 daily sessions, each of which consisted of 3 independent trials which were separated by at least 10-minute intervals. The data displayed represent the average fall latency on the 3rd day of testing. To test overall locomotor activity, we performed open field testing in which mice were placed in the center of a 45×45 cm chamber and locomotor activity was recorded by an overhead webcam connected to a laptop computer, and animals' movements were tracked for 30 minutes using ANY-Maze. The data displayed represent the total locomotor activity during the 30-minute period. We also tested sensorimotor behaviors using the plantar tape test (also known as the adhesive removal test). Briefly, several days prior to the experiment, mice were acclimated to handling (scruffing). On the day of the experiment, mice were acclimated to a standard mouse cage without bedding for 60s. Mice were then gently immobilized, and a 3×4 mm tape was firmly applied to the plantar surface of the left hindpaw such that it covered the glabrous skin. The mice were returned to the testing cage, and the latency (in seconds) to remove the tape was recorded by an experimenter blinded to the genotypes of the animals.

Conditioned place preference (CPP) assay: To test whether STING agonists would induce CPP in naïve mice, we used a three repeated-pairing conditioning protocol, as a single trial was found to be insufficient to induce place preference in the morphine control group. To test whether STING agonists could induce CPP in chronic pain (CIPN and BCP) models, a measure of ongoing or spontaneous pain, we used a single-trial pairing protocol, as this was sufficient to induce robust CPP in the clonidine control group. These observations are consistent with previous reports. All mice underwent 3 d of preconditioning habituation in a temperature- and humidity-controlled room under low-light conditions between 08:00-16:00 in a two compartment CPP chamber (Med Associates Inc.). The two compartments differed by both visual and tactile cues. On the 4th day, animal behavior was video-recorded using a webcam connected to a laptop computer, and animal movement was automatically tracked for 15 minutes using the ANY-Maze software (Stoelting). Baseline recordings revealed that mice generally formed a slight preference to one chamber. Mice exhibiting strong preference (>80% of time spent in one chamber) were not used for the experiments. On the conditioning day(s) (day 5 for single trial; day 5-7 for repeated trials), two pairing sessions were performed, with vehicle pairing in the AM session and drug treatment (e.g., vehicle or drug) in the PM session, separated by 4 h. In the AM session, mice always received vehicle (10 μl saline, delivered via i.t. injection under isoflurane anesthesia) and were immediately placed in their preferred CPP chamber for 2 h. In the PM session, mice received drug treatment (10 μl, delivered via i.t. injection under brief isoflurane anesthesia) and were placed in their non-preferred chamber immediately (PBS, morphine, and clonidine) or 4 h later (DMXAA, ADU-S100), time points corresponding to the peak antinociceptive effects of these agents. 24 h after the completion of the pairing experiments, mice were placed in the CPP test box with access to both chambers and behavior was recorded for 15 minutes and analyzed using the ANY-Maze software for chamber preference. CPP score was calculated as the inverse of the time spent in the preferred chamber according to: [post-preference (sec)—pre-preference (sec)]. Vehicle served as a negative control for these experiments, while morphine (three trials/naïve mice) or clonidine (single trial/ongoing pain) served as positive controls.

Conditioned place aversion (CPA) assay: A modified CPA assay was used based on a previous report. Briefly, male and female mice lacking Ifnar1 or STING (and their wildtype littermates) were habituated for three days (30 minutes each) to a small custom-designed two-chamber CPA apparatus which was placed on an elevated mesh rack. Each chamber contained unique visual cues (black and white cross-hatching or plain white walls) and measured approximately 4.5×9 inches across. On the final day of habituation, baseline (BL) preferences were video-recorded for 10 minutes and movement was tracked using the ANY-Maze software. Following BL measurements, animals were sequestered to their preferred chamber and pairing was conducted by repeatedly stimulating the left hindpaw once every 10 seconds for 10 minutes using a 0.04 g filament, which is normally innocuous and does not elicit withdrawal responses or CPA in naïve mice. No mice exhibited greater than 65% preference for either chamber, and thus, all mice in which BLs were recorded were used in the experiment. After pairing, mice were returned to their home cage for 20 minutes, after which time they were returned to the CPA chamber with equal access to both chambers. Post-pairing behaviors were video recorded for 10 minutes and movement was again tracked using ANY-Maze software. CPA score was calculated as the time spent in the paired chamber according to: [post-preference (sec)—pre-preference (sec)].

Behavioral testing in non-human primates: Five adult rhesus monkeys (Mucaca mulatta, 9.3-13.8 kg) were maintained in an AAALAC-approved facility at Wake Forest University School of Medicine in accordance with Wake Forest University IACUC regulations. Animals were individually housed in temperature- and humidity-controlled species-specific rooms maintained in a 12 h light/12 h dark cycle. All animals have been previously trained in the tail-withdrawal assay. All experimental procedures were conducted in accordance with the Guide for the Care and Use of Laboratory Animals standards adopted by the NIH. Drug infusion was through a previously implanted spinal catheter. Behavioral testing was performed by an experimenter who was blinded to the drug being administered. A cold-water tail-withdrawal assay was used to evaluate nociceptive responses to 16° C. water with a cut-off of 20 sec. 0.3 mL of 2% menthol gel (Millipore Sigma) was applied to the tail for 3 minutes to induce transient and reversible cold allodynia. The antinociceptive effects of ADU-S100 were measured at different time points i.e., before and 5 h, 24 h, and 29 h after intrathecal administration.

Culture of LLC cells and measurement of local tumor burden: Murine LLC cells were cultured in high glucose (4.5 g/L) Dulbecco's Modified Eagle Medium (DMEM; ThermoFisher) supplemented with 10% fetal bovine serum (Gibco, ThermoFisher) and 1% Antibiotic-Antimycotic solution containing penicillin, streptomycin, and Amphotericin B (ThermoFisher, 15240062). Cells were cultured in the presence of 5% CO2 at 37° C. Cells were passaged using a 0.05% trypsin digest once they achieved 70% confluency. Prior to in vivo injection, cells were harvested with 0.05% trypsin digestion followed by several washes in PBS. Cell counts were performed using a hemocytometer and diluted to achieve a suspension of 1×10⁸ ml-1 cells in PBS. To determine whether i.t. STING agonists altered local tumor burden induced by femoral inoculation with LLC cells, mice were lightly anesthetized with isoflurane at the conclusion of the experiment, followed by measurement of the total circumference of the widest aspect of the thigh, which accurately reflects tumor burden. Ratios were computed between the ipsilateral (LLC-inoculated) and contralateral (tumor-free) legs.

Drug delivery: All drugs were dissolved in sterile saline or PBS, which was used as the corresponding vehicle control, with the exception of DMXAA, which was first dissolved in PBS containing 0.75% NAHCO₃ and was further diluted 10-fold prior to experimentation, and H-151 and C-176, which were diluted in PBS containing 5% DMSO and 0.0625% Tween-20 due to poor solubility. For the experiments utilizing i.t. 2′3′-cGAMP and 3′3′-cGAMP complexed with Lipofectamine 2000 (ThermoFisher, 11668), 10 μg 2′3′-cGAMP or 3′3′-cGAMP was incubated at room temperature for 30 minutes in sterile PBS with 3 μl Lipofectamine 2000. In this case, the vehicle control was 3 μl Lipofectamine 2000 in PBS, as in a previous study which used this approach. Drug concentrations and route of administration are generally provided in the figures or figure legends. DMXAA and ADU-S100 were generally used at a concentration of 35 nmol (i.t.), administered via two injections 24 h apart. For experiments utilizing rabbit anti-IFN-α or -β neutralizing antibodies, a polyclonal rabbit IgG antibody served as the control. Antibodies were utilized in vivo at 300 ng/mouse or in vitro at 300 ng/ml. Paclitaxel was administered via repeated i.p. injections every other day for 4 days at a dose of 2 mg/kg. For the whole mount DRG recordings, young mice (4-6 weeks) were administered a single 6 mg/kg dose one week prior to the experiment. All intrathecal injections were performed under brief anesthesia with 2-2.5% isoflurane. Drugs administered by intrathecal injection were preceded by shaving a small area on the back corresponding to the injection site, followed by spinal puncture using a 30-gauge needle between the L5-L6 levels to deliver a maximum of 5-10 μl into the cerebrospinal fluid. Successful intrathecal injection was always confirmed by a brisk tail-flick upon delivery. For the experiments involving intraplantar (i.pl.) injection, IFN-α was dissolved in sterile saline and administered at the concentrations indicated in the figures in a total volume of 20 μl, administered under 2-2.5% isoflurane anesthesia to minimize animal stress. For these experiments, vehicle (saline) or IFN-α were slowly and carefully expressed into the left dorsal hindpaw of anesthetized mice and allowed to perfuse the tissue for ˜20 seconds prior to removal of the needle. Mice were monitored for ˜30 seconds after removal of the needle to ensure that the solution did not express from the tissue. Mechanical sensory testing was done anteriorly and care was taken to avoid Von Frey filament stimulation of the injection site.

ELISA: Mouse high-sensitivity IFN-α ELISA kit (42115-1) and IFN-β ELISA kit (42410-1) were purchased from PBL Assay Science and performed on serum, DRG lysate, or cell culture medium according to the manufacturer's instructions. Serum was obtained from whole blood through cardiac puncture at the time of euthanasia, followed by incubation for 30 minutes at 35° C. to promote clotting. Subsequently, samples were centrifuged at 4° C. at 2,500 xg for 15 minutes, followed by careful isolation of serum (supernatant). For ELISA on cell culture medium, samples were first concentrated approximately 10-fold using Amicon centrifugal filters (Millipore Sigma, UFC8030). DRGs were lysed by mechanical homogenization in a pH 7.4 CHAPS lysis buffer (FivePhoton Biochemicals, CIB-1-7.4-60) supplemented with a protease inhibitor cocktail (Millipore Sigma, 11697498001) and phosphatase inhibitor cocktail (FivePhoton Biochemicals, PIC1). For DRG lysate, Pierce BCA (ThermoFisher, 23225) assays were performed according to the manufacturer's instructions to quantify protein concentration, and ELISA results are reported normalized to protein concentration (pg/mg DRG tissue). To measure IFN-β levels in cerebrospinal fluid (CSF) isolated by spinal catheter from NHPs, we utilized a cynomolgus IFN-β ELISA kit (PBL Assay Science, 46415-1), which was performed using 50 μl CSF according to the manufacturer's instructions. Standard curves were performed in all experiments and values interpolated using GraphPad Prism version.

In situ hybridization (ISH): Mice were deeply anesthetized with isoflurane and transcardially perfused with 25 ml PBS followed by 25 ml 4% paraformaldehyde/1% picric acid. Following perfusion, L3-L5zen spinal cords or DRGs were isolated and post-fixed overnight at 4° C. in the same fixative. Tissues were subsequently washed several times in PBS, followed by cryopreservation using a sucrose gradient. tissues were then embedded in OCT medium (Tissue-Tek) and cryosectioned to produce 14 μm-thick sections, which were mounted onto charged slides. Each tissue block (and thus, each slide) contained both WT and KO tissues to account for any variability in staining between slides, and to control for the specificity of RNAscope probes targeting STING (Tmem173) or Ifnar1. In situ hybridization was performed using the RNAscope system (Advanced Cell Diagnostics) in accordance with the manufacturer's instructions, using a protocol tailored to the Multiplex Fluorescent Kit v2. We used probes directed against murine Ifnar1 (catalog 512971, NM_010508.2) and murine STING (Tmem173; catalog 413321, NM_028261.1). Following the completion of the RNAscope protocol, immunohistochemistry was performed as described in the next section. The in situ hybridization experiments were repeated in three independent experiments.

Immunohistochemistry and imaging: Adult mice were deeply anesthetized with isoflurane and perfused through the ascending aorta with 20-25 ml PBS, followed by 4% paraformaldehyde/1% picric acid. Following perfusion, L3-L5 DRGs, L3-L5 spinal cord segments, or mouse plantar hindpaw skin were removed and post-fixed in the same fixative overnight. Tissues were cryopreserved and sectioned in the same manner described for ISH. Sections were blocked for 1 h at room temperature in a solution containing 1% BSA, 0.1% triton X-100, 5% NDS, and mouse-on-mouse blocking reagent (if mouse antibodies were used) in accordance with the manufacturer's instructions. Sections were stained in a humidified chamber overnight at 4° C. with the following primary antibodies: anti-GFAP (mouse, 1:2000, Millipore Sigma, MAB360), Iba1 (rabbit, 1:500, Wako, 019-19741), anti-β Tubulin III (mouse, TuJ1; 1:1000, Millipore Sigma, T8578), anti-CGRP (guinea pig, 1:1000, Peninsula, T-5027), and anti-GFP (chicken, 1:2000, Abcam, ab13970). The next day, sections were washed several times, followed by incubation with Isolectin GS-IB4-488 conjugate (1:500, ThermoFisher, 121411), Nissl/NeuroTracer-640 (1:200, ThermoFisher, N21483), or species-specific secondary antibodies conjugated to -488, -555, or -633 fluorophores purchased from Biotium, which were raised in donkey (1:400), stained in a humidified chamber overnight at 4° C. Sections were subsequently washed and coverslipped using a few drops of DAPI Fluoromount-G mounting medium (Southern Biotech, 0100-20). Stained sections were examined using a Nikon fluorescent microscope, and images were captured with a mounted CCD Spot camera. For high resolution imaging, images were also captured using a Zeiss LCM 880 confocal microscope with a z-step size of 1 μm, with 10-12 steps. For images captured on the confocal microscope, maximum projections were produced using the Zeiss ZEN software package (v3.2). Images directly comparing two groups (e.g., WT and KO mice) were taken using the same acquisition settings.

Quantification of total cell numbers and innervation density: Quantification of total neuron numbers was performed by counting TuJ1+ neurons with DAPI+ nucleoli in the L5 DRG, counting every third section. To quantify immunostaining in the superficial dorsal horn (IB4/CGRP density for innervation staining; GFAP, Iba1, and DAPI for quantification of glial cell activation), the Image J plugin Fiji was utilized to draw a box narrowly around the relevant regions followed by quantification of pixel density by an experimenter blinded to the treatment groups. For the quantification of CGRP and IB4 in the SDH, the data reported (normalized density) represent the pixel intensity of each signal normalized to the area, reported as arbitrary units (A.U.). For the quantification of glial cell reaction, values represent the ratio of pixel density for each channel on the ipsilateral (SNI-injured) side normalized to the contralateral (uninjured) side. To quantify the density of nerve fibers in the glabrous skin of the hindpaw, we counted the number of TuJ1+, CGRP+, or IB4+ nerve fibers (>5 μm in length), in 3-5 separate sections for each animal. Data display the average number of nerve fibers positive for each marker per mm³. In all cases, the images were quantified by an experimenter blinded to animal genotypes.

Whole-cell patch clamp recordings in dissociated mouse DRG neurons: DRGs were removed under sterile conditions from 4-6-week-old male and female mice and digested with a collagenase (1.25 mg/ml; Roche)/dispase-II (2.4 U/ml; Roche) solution for 90 minutes at 37° C., followed by incubation in 0.25% trypsin for 10 minutes at 37° C. Cells were mechanically dissociated with a flame-polished Pasteur pipette and plated in a 50 μl bead for 30 minutes prior to flooding the chamber. Cells were plated on glass cover slips coated with 0.5 mg/ml poly-D-lysine (Millipore Sigma, A-003-E) and cultured in a Neurobasal medium (Gibco, ThermoFisher) supplemented with 10% FBS, 2% B27 supplement (Gibco, ThermoFisher), and 1% penicillin-streptomycin (Gibco, ThermoFisher) at 37° C. in 5% CO2 for 24 h prior to recordings. Coverslips were transferred to a 300 μl recording chamber continuously perfused (˜3 ml/min) with ACSF where small-diameter DRG neurons (<25 μm) could be identified using a 40× water-immersion objective on an Olympus BX51WI microscope. Whole-cell patch-clamp configuration was made, and current clamp mode was performed to record action potentials. The action potentials were evoked by current injection steps from 0-130 pA with an increment of 10 pA in 600 ms. Rheobase was measured by injecting currents from 0 pA with an increment of 10 pA in 30 ms. Patch pipettes were pulled from borosilicate capillaries (Chase Scientific Glass Inc.) and filled with a pipette solution containing (in mM): 126 potassium gluconate, 10 NaCl, 1 MgCl₂, 10 EGTA, 2 Na-ATP, and 0.1 Mg-GTP, adjusted to pH 7.3 with KOH. The external solution was composed of (in mM): 140 NaCl, 5 KCl, 2 CaCl₂, 1 MgCl₂, 10 HEPES, 10 glucose, adjusted to pH 7.4 with NaOH. The resistance of pipettes was 4-5 MΩ. Series resistance was compensated for (>80%) and leak subtraction was performed. Data were low-pass filtered at 2 KHz and sampled at 10 KHz. Data were recorded and analyzed using the pClamp10 (Axon Instruments) software. Recordings from DRG neurons (<25 μm) were performed between 24-48 h of plating. The vehicle or IFN-I was perfused for 3 min, and APs and rheobase were recorded before (control) and 4-5 min after the onset of the perfusion of the vehicle or IFN-I.

Primary culture and whole cell patch clamp recordings from NHP DRG neurons: Lumbar DRGs were isolated from disease-free NHPs and delivered on ice within 4 h of death. Neurons were dissociated, plated, and cultured as described for mouse DRG neurons. 24 h after plating, whole-cell patch clamp recordings were performed on small-diameter DRG neurons (<50 μm) at room temperature using patch pipettes with a resistance of 2-3 MΩ. Whole-cell patch-clamp configuration was made, and current clamp mode was performed to record action potentials. The action potentials were evoked by current injection steps from 0-580 pA with an increment of 20 pA in 1500 ms. Rheobase was measured by injecting currents from 0 pA with an increment of 15 pA in 60 ms. The experimental setup and data recording were performed as in the section detailing patch clamp recordings on mouse DRG neurons. The vehicle or IFN-I was perfused for 3 min, and APs and rheobase were recorded before (control) and 4-5 min after the onset of the perfusion of the vehicle or IFN-I.

Primary culture and whole cell patch clamp recordings from human DRG neurons: Non-diseased human DRGs (hDRGs) were obtained from three donors through NDRI (Philadelphia, Pa.) with permission of exemption from Duke IRB. Postmortem lumbar hDRGs were delivered in ice-cold culture medium within 48-72 hours of death. Upon delivery, hDRGs were rapidly dissected from nerve roots and minced in a calcium-free HBSS. hDRGs were digested at 37° C. in a humidified 5% CO₂ incubator for 120 min with a collagenase (1.25 mg/ml; Roche)/dispase-II (2.4 U/ml; Roche) solution in HBSS. hDRGs were mechanically dissociated using fire-polished pipettes, passed through a 100 μM nylon mesh filter, and centrifuged (500 xg for 5 min). Cells were resuspended, plated on 0.5 mg/ml poly-D-lysine-coated glass coverslips, and grown in culture medium identical to mouse and NHP DRGs. Patch clamp recordings were conducted in small-diameter (<55 μm) hDRG neurons. While the condition of hDRG neurons generally did not permit consistent recording of evoked action potentials, we recorded changes in membrane potential following perfusion with vehicle or IFN-I (30 U/ml), conducted as in the section detailing patch clamp recordings on mouse DRG neurons.

Whole-cell patch clamp recordings in whole-mount DRGs ex vivo: To test the effects of anti-IFNβ neutralizing antibodies, naïve male and female mice were utilized. For the experiment testing the effects of ADU-S100 in Ifnar1+/+ or Ifnar1−/− mice following PTX treatment, male and female mice of the indicated genotypes were treated with a single dose of 6 mg/kg PTX followed by recording 1 week later. To perform whole-cell patch clamp recordings in whole-mount DRGs, L1-L5 DRGs were carefully isolated and placed in oxygenated ACSF. Using a stereoscope, peripheral and central DRG projections and connective tissue were dissected away. DRGs were lightly digested for 20 minutes using an enzymatic mixture consisting of 0.32 ml collagenase A (1 mg/mL) and Trypsin (0.25%). Intact DRGs were then incubated in ACSF oxygenated with 95% O₂ and 5% CO₂, supplemented with the relevant treatment: (1) 300 ng/ml rabbit polyclonal IgG control or rabbit anti-IFnβ for 2-3 hours at 37° C.; or (2) vehicle (PBS) or 30 μM ADU-S100 in PBS for 2-3 hours at 37° C. Following incubation, DRGs were transferred to a recording chamber, where neurons could be visualized using a 40× water-immersion objective on an Olympus BX51WI microscope. The recording chamber was continuously perfused (2-3 ml/min) with ACSF. The pipette solution contained 140 mM KC1, 2 mM MgCl2, 10 mM HEPES, 2 mM Mg-ATP at pH 7.4. The resistance of the glass pipettes was 8-10 MΩ. Data were acquired and analyzed using the pClamp10 (Axon Instruments) software.

Whole cell patch clamp recording of sodium and calcium currents in vitro: For sodium current recordings, HEK-hNav1.7 stable cell line was purchased from SB Drug Discovery (Glasgow, United Kingdom). Cells were cultured in MEM containing 10% FBS, 1% streptomycin/penicillin, L-glutamine (2 mM) and blasticidin (0.6 mg/ml). Whole-cell patch-clamp recordings on HEK293 cells and DRG neurons were conducted at room temperature according to the protocol for whole cell patch clamp in dissociated DRG neurons. In voltage-clamp experiments, transient Na+ currents were evoked by a test pulse to 0 mV from the holding potential (−70 mV). Calcium current was evoked in DRG neurons by applying a 40-ms step depolarization to −10 mV from −80 mV. For calcium recordings, the external solution contained the following: 135 mM TEA-Cl, 1 mM CaCl₂, 10 mM HEPES, 4 mM MgCl₂ and 0.1 μM TTX, adjusted to a pH of 7.4 with TEA-OH. The pipette solution contained 126 mM CsCl, 5 mM Mg-ATP, 10 mM EGTA and 10 mM HEPES, adjusted to a pH of 7.3 with CsOH.

Whole-cell patch-clamp recording in mouse spinal cord slices: 4 to 6-week-old mice were anesthetized with urethane (1.5-2.0 g/kg, i.p.). The lumbosacral spinal cord was quickly removed and placed in ice-cold dissection solution (240 mM sucrose, 25 mM NaHCO₃, 2.5 mM KCl, 1.25 mM NaH₂PO₄, 0.5 mM CaCl₂, and 3.5 mM MgCl₂), equilibrated for at least 30 minutes with 95% O2 and 5% CO₂. After extraction of the spinal cord under anesthesia, animals were rapidly euthanized via decapitation. Transverse spinal slices (300 to 400 μm) were prepared using a vibrating microtome (VT1200S Leica). The slices were incubated at 32° C. for at least 30 min in ACSF equilibrated with 95% O₂ and 5% CO₂. The slices were placed in a recording chamber and completely submerged and perfused at a flow rate of 2 to 4 ml/min with ACSF saturated with 95% O₂ and 5% CO₂ at room temperature. Lamina II neurons in lumbar segments were identified as a translucent band under a microscope (BX51WIF; Olympus) with light transmitted from below. Whole-cell voltage-clamp recordings were made from outer lamina II neurons using patch pipettes. The patch pipette solution used to record sEPSCs contained 135 mM K-gluconate, 5 mM KCl, 0.5 mM CaCl₂, 2 mM MgCl₂, 5 mM EGTA, 5 mM HEPES, and 5 mM Mg-ATP (pH 7.3 adjusted with KOH). The patch pipettes had a resistance of 8 to 10 M. The sEPSC recordings were made at a holding potential (VH) of −70 mV in the presence of 10 μM picrotoxin and 2 μM strychnine. mEPSCs were recorded in the presence of 0.5 μM TTX, perfused 4 min prior to drug application. Signals were acquired using an Axopatch 700B amplifier. The data were stored and analyzed with a PC using pCLAMP 10.3 software.

Data collection, statistics, and transparency: All data are expressed as the mean±s.e.m., as indicated in the figure legends. In most cases, each data point corresponds to an individual animal. For electrophysiology data, each data point corresponds to an individual neuron, with neurons from at least 3 separate animals/subjects analyzed in all cases. The sample sizes utilized in each experiment were based on our previous studies. All data were included in the analyses (no outliers removed). In some cases, indicated within the figure legends, sample sizes are larger than would be necessary to observe differences between groups because experiments were repeated multiple times to ensure reproducibility with all relevant controls. All experimental data shown has been reliably reproduced by multiple lab members. All statistical analyses were completed using GraphPad Prism. Unless otherwise noted in figure legends, behavioral and biochemical data and some electrophysiological data were analyzed using two-way ANOVA with Dunnett's post-hoc test to account for multiple comparisons. For the CPP assay, each experimental group was compared with the vehicle treatment group using one-way ANOVA with Fisher's least significant difference post-hoc test. A two-tailed t-test was utilized to compare two groups when analyzing sensory neuron numbers and innervation densities. Electrophysiological data were tested using two-tailed t-test (for two groups) or two-way ANOVA (for multiple time points) with Dunnett's post hoc test. The criterion for statistical significance was defined as p<0.05. Throughout this manuscript, asterisks correspond to the following significance levels: * p<0.05, ** p<0.01, *** p<0.001, **** p<0.0001. For full transparency, all raw data is included in the Supplementary Notes in Donnelly, et al. (“STING controls nociception via type I interferon signalling in sensory neurons.” Nature 591, 275-280 (2021)).

### Example 2. STING Controls Nociception via Type I Interferon Signaling in Sensory Neurons

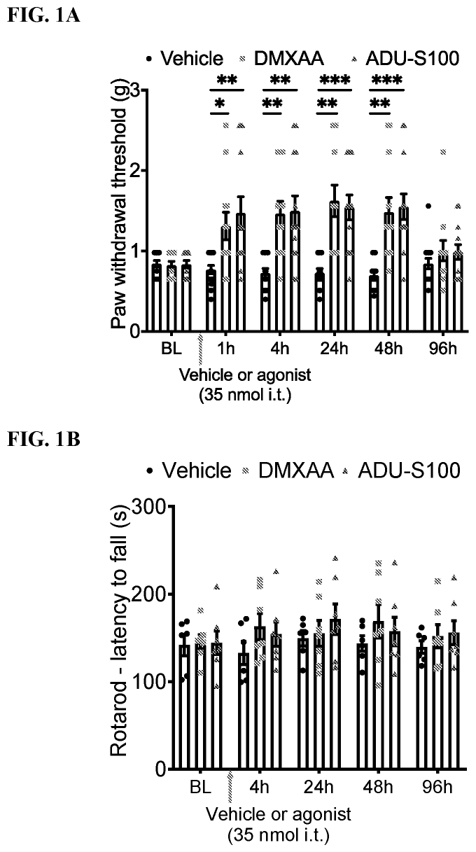

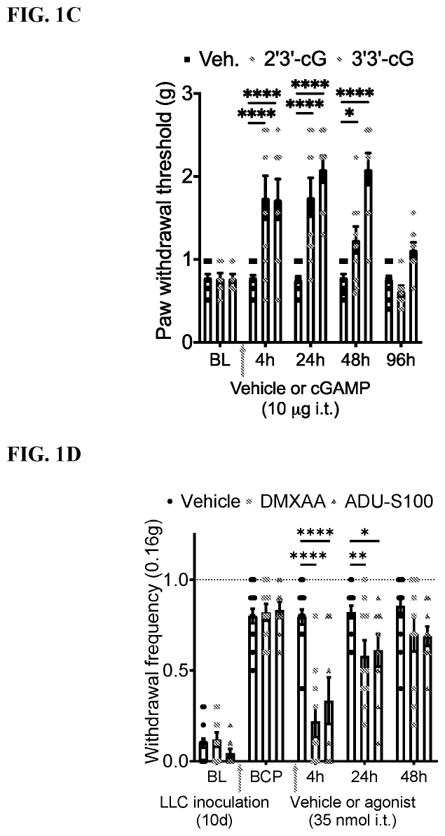

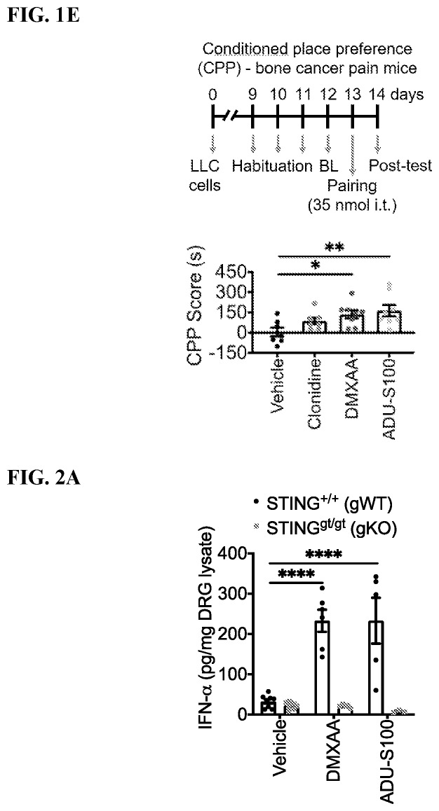

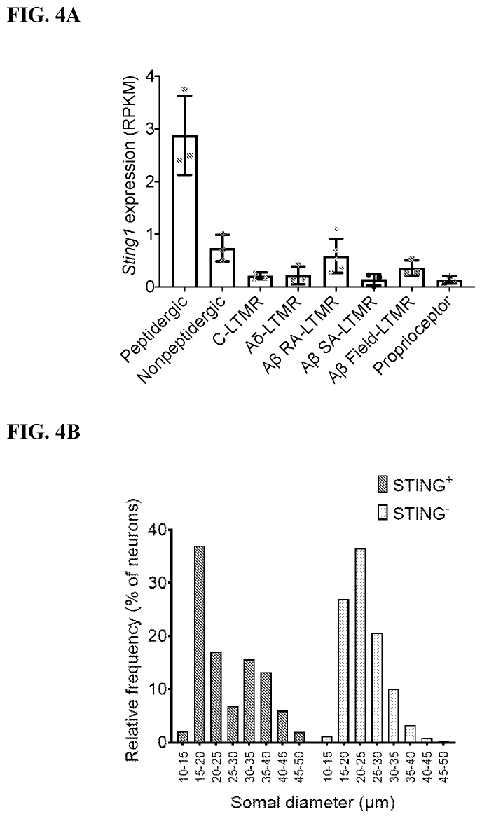

Nociception, or the detection of noxious stimuli, is a highly evolutionarily conserved system . Although peripheral nociceptors were classically thought to serve as simple sensors of hazardous stimuli, present to elicit withdrawal and future avoidance of pain-evoking stimuli, nociceptors are now recognized as critical regulators of inflammation and immunity. Nociceptors can be directly activated by damage- or pathogen-associated molecular patterns (DAMPs or PAMPs) produced by invading pathogens or damaged host cells, evoking pain, itch, or analgesia. Deep sequencing of dorsal root ganglion (DRG) sensory neurons demonstrates that nociceptors express a large array of DAMP or PAMP-sensing pattern recognition receptors (PRRs) canonically expressed by immune cells and associated with host defense. We observed that STING (recently renamed STING1, or stimulator of interferon response cGAMP interactor-1), an endoplasmic reticulum-bound DNA sensor, is highly expressed in nociceptors (FIG. 4A), which we confirmed by in situ hybridization (FIG. 4B). Given this pattern of expression and the variety of mediators that act through STING (Extended Data FIG. 1d of Donnelly, supra), we hypothesized that STING may regulate nociception.

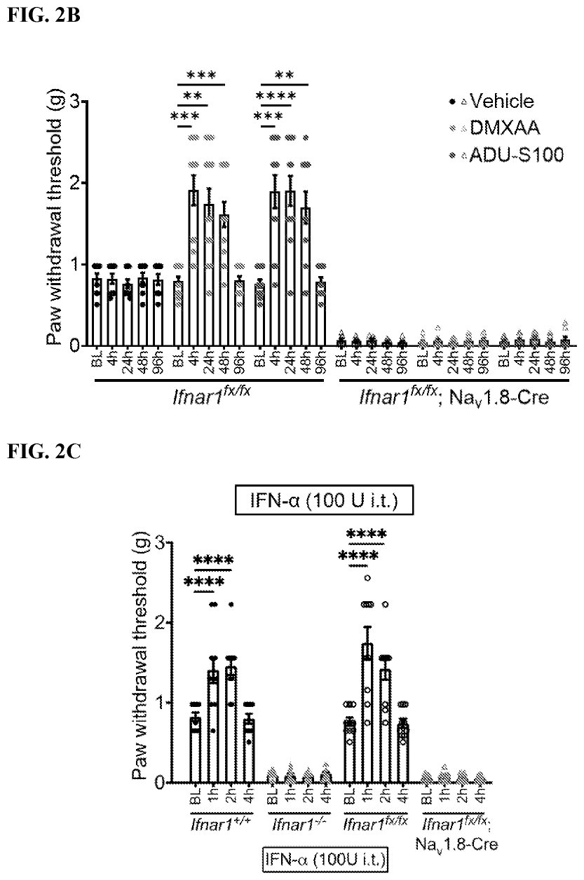

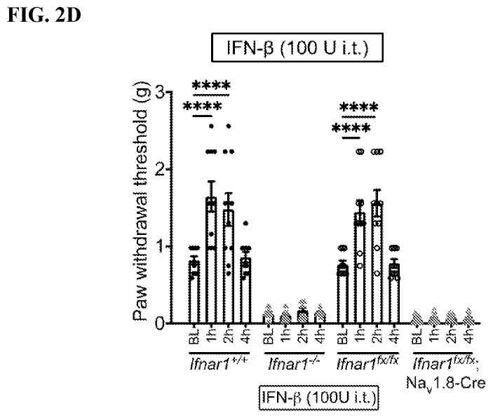

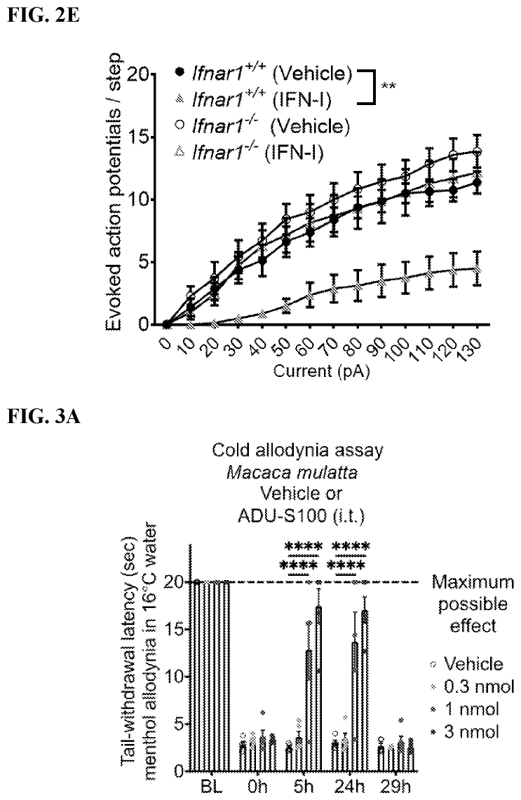

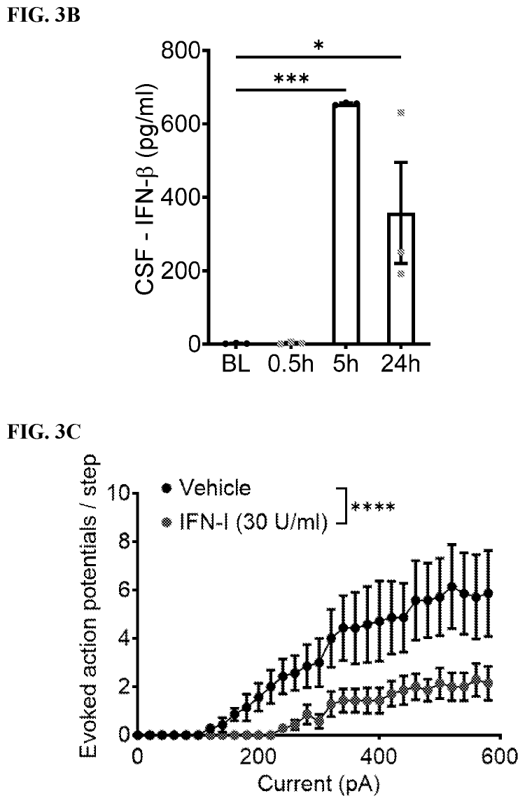

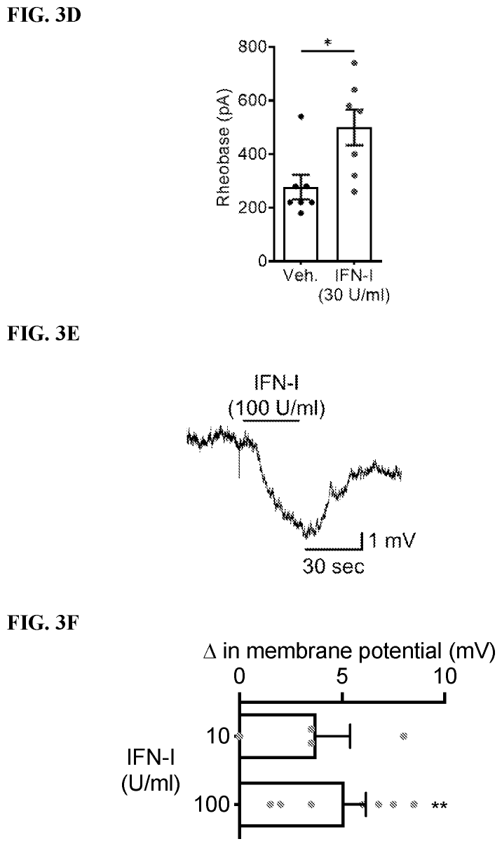

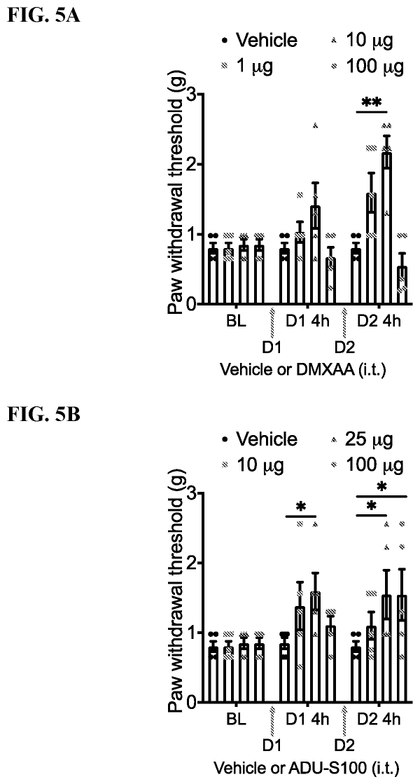

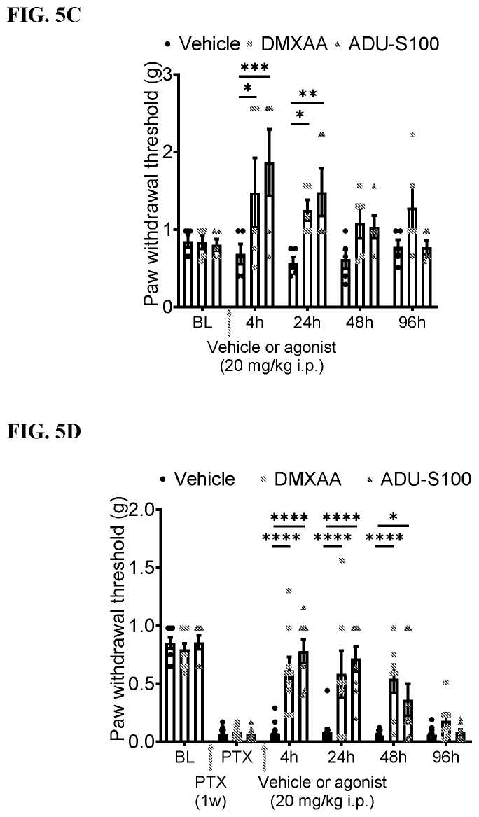

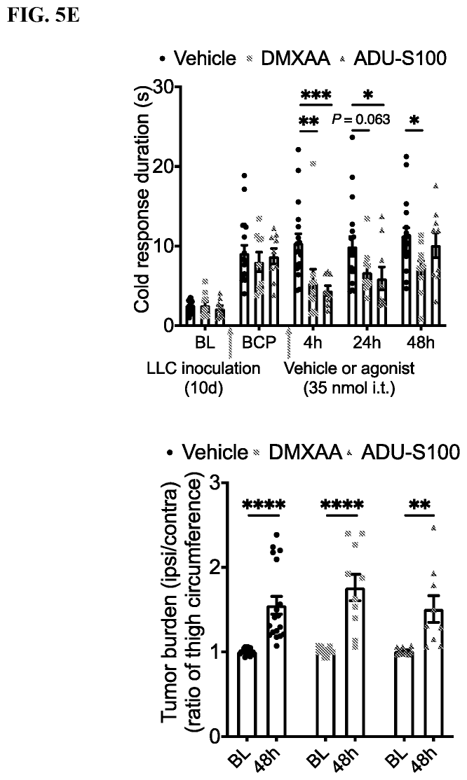

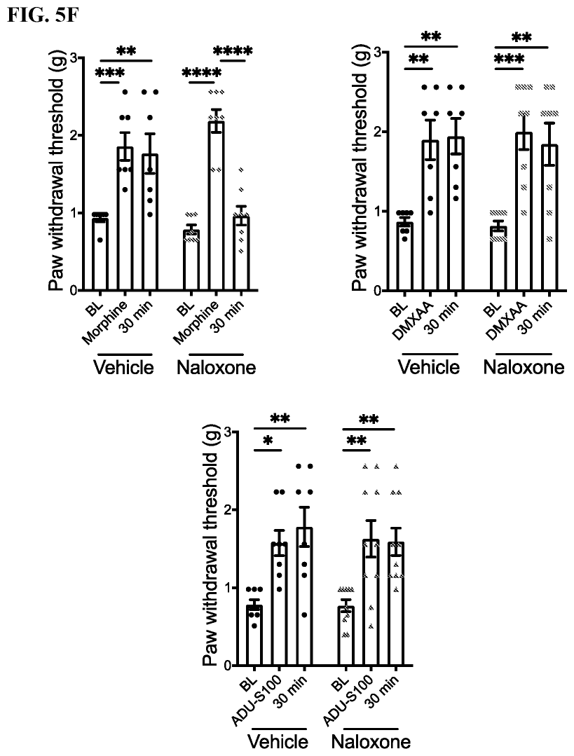

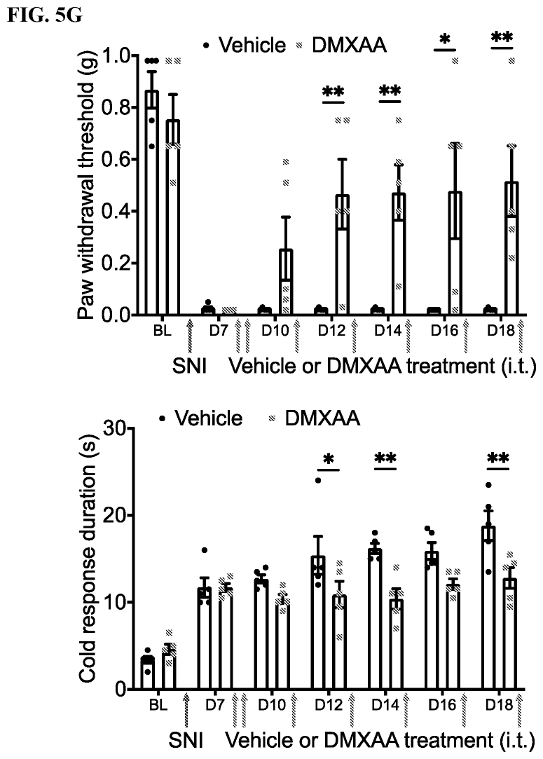

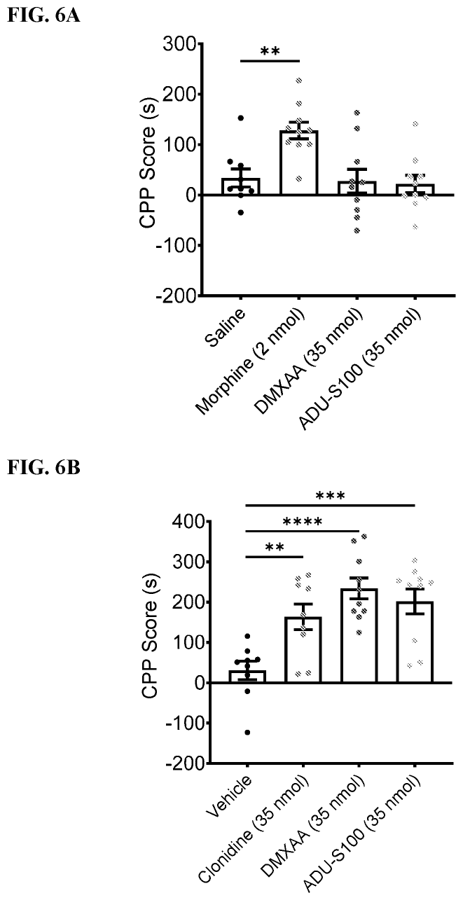

STING agonists suppress nociception: To determine whether STING regulates nociception, we administered synthetic (DMXAA, ADU-S100) or natural (2′3′-cGAMP, 3′3′-cGAMP) STING agonists to naïve mice via intrathecal (i.t.) injection, thereby targeting cells in the spinal cord and DRG. Activation of STING induced dose-dependent antinociception in naïve mice, elevating mechanical sensory thresholds for up to 48 h without impairing motor coordination (FIG. 1A-C). We next tested the analgesic potential of these agonists in a chemotherapy-induced peripheral neuropathy (CIPN) model established using paclitaxel (PTX) and observed that intrathecal or systemic delivery of STING agonists could reverse mechanical and cold allodynia (FIG. 5D), hallmarks of neuropathic pain. STING agonists also attenuated neuropathic pain after nerve injury (Extended Data FIG. 2i-j of Donnelly, supra). Given the role of STING in augmenting antitumor immunity, we tested whether STING agonists could suppress pain in a mouse model of bone cancer pain (BCP; FIG. 1D), a particularly severe and insufficiently controlled pain condition, and found that i.t. STING agonists could rapidly suppress cancer-induced mechanical and cold allodynia without changing local tumor burden (FIG. 1D; FIG. 5E). To assess ongoing pain, we also performed conditioned place preference testing (CPP; Extended Data FIG. 3a of Donnelly, supra). In naïve mice, i.t. morphine, but neither DMXAA nor ADU-S100 could induce place preference (FIG. 6A), suggesting STING agonists do not activate the addiction and reward circuitry of the brain like morphine and other opioids. To test whether DMXAA and ADU-S100 could suppress ongoing pain, we performed CPP testing in mice with CIPN or BCP and found STING agonists induced CPP in both models (FIG. 6B; FIG. 1E). We also tested whether the opioid receptor antagonist naloxone could reverse the antinociceptive effects of STING agonists and found that, while naloxone reversed morphine-mediated antinociception, STING agonist-mediated antinociception was not affected by naloxone (FIG. 5F). Furthermore, repeated i.t. administration of DMXAA to naïve mice or mice following spared nerve injury (SNI) did not show signs of tolerance (FIG. 5G). Rather, repeated administration of DMXAA attenuated SNI-induced astrogliosis (Extended Data FIG. 2t of Donnelly, supra), a critical contributor to many pathological pain states.

STING regulates steady-state nociception: We also analyzed pain sensitivity in STING “goldenticket” mice which lack STING signaling globally (STING-gKO; STINGgt/gt). Strikingly, we observed that these mice had dramatically increased sensitivity to mechanical and cold stimuli (FIG. 1i-k of Donnelly, supra). To confirm these results, we utilized a conditioned place aversion (CPA) assay in which mice were stimulated repeatedly in a preferred chamber with a very low threshold filament (0.04 g) that is largely imperceptible to naïve mice 16, which resulted in CPA in STINGgt/gt but not STING+/+ mice (FIG. 1l-n of Donnelly, supra). DRG nociceptors cultured from STINGgt/gt mice also exhibited increased action potential firing and decreased rheobase (Extended Data FIG. 3f-j of

Donnelly, supra), suggesting loss of STING produces nociceptor hyperexcitability. Notably, mice lacking STING exhibited no changes in peripheral or central innervation density, total DRG neuron numbers, or sensorimotor behaviors (Extended Data FIG. 4a-n of Donnelly, supra). Additionally, mice lacking STING selectively in peripheral sensory neurons (STINGfx/fx; Nav1.8-Cre mice; STING-cKO)17 revealed a similar phenotype (FIG. 1i-k of

Donnelly, supra), suggesting these effects are owed to sensory neuron-intrinsic STING signaling. In addition, naïve mice injected with small molecule inhibitors of STING (H-151, C176) exhibited transient dose-dependent hypersensitivity (Extended Data FIG. 5a-d of Donnelly, supra). STING agonists did not produce antinociception in STINGgt/gt mice, but reduced mechanical allodynia at later timepoints in STING-cKO mice (Extended Data FIG. 5e-h of Donnelly, supra), indicating that neuronal STING is required for the initial antinociceptive effects, but other cell types may contribute to the long-lasting effects. We also tested whether the antinociceptive effects of STING agonists remained intact in naïve mice in which TRPV1+ nociceptors were ablated using resiniferatoxin (RTX), as these neurons exhibit the highest expression of STING (FIG. 4A). RTX treatment led to heat insensitivity in the hot plate test and abolished the antinociceptive effects at early timepoints, while muted but statistically significant effects were retained at later timepoints (Extended Data FIG. 5i-k of Donnelly, supra). To test whether adaptive immune cells important for STING agonist-mediated antitumor immunity 3 contribute to these effects, we administered STING agonists to Prkdcscid mice, which lack mature B and T cells, and observed an unaltered time course of STING agonist-mediated antinociception (Extended Data FIG. 5l of Donnelly, supra). Thus, sensory neuron-intrinsic STING signaling regulates steady-state nociception during homeostasis (Extended Data FIG. 5m of Donnelly, supra), but STING signaling in multiple cell types may contribute to the prolonged antinociceptive effects of STING agonists.

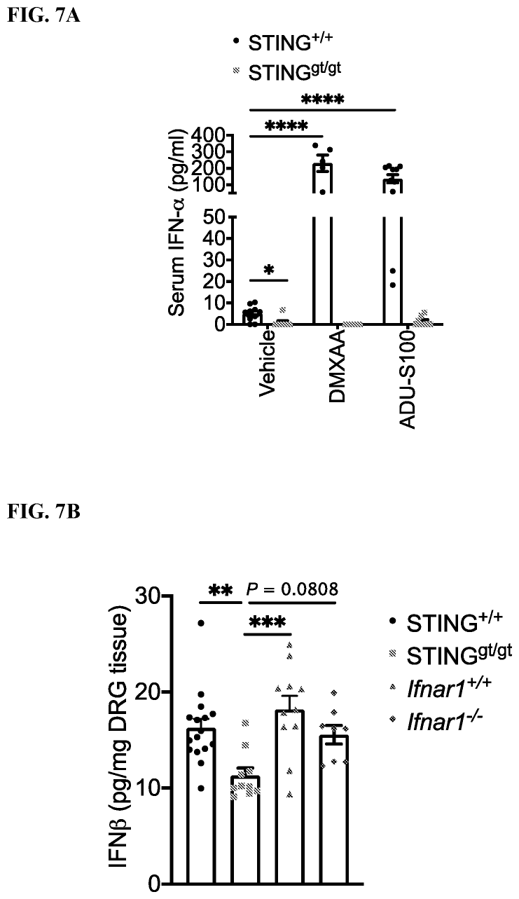

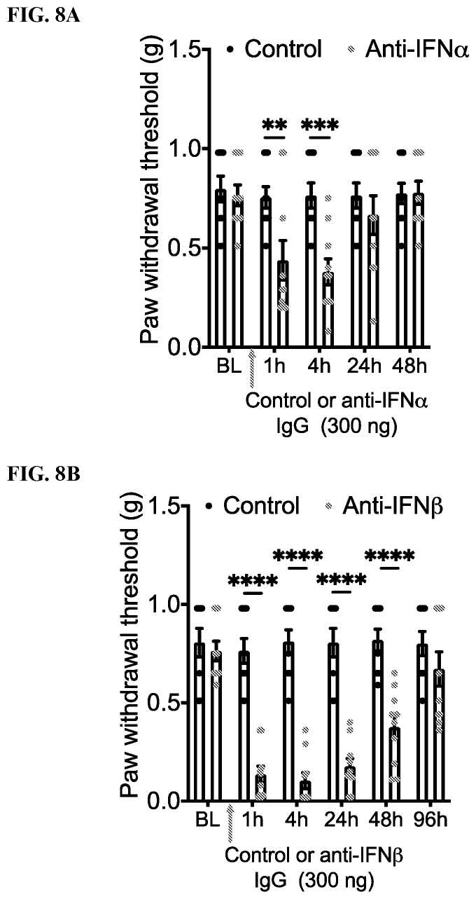

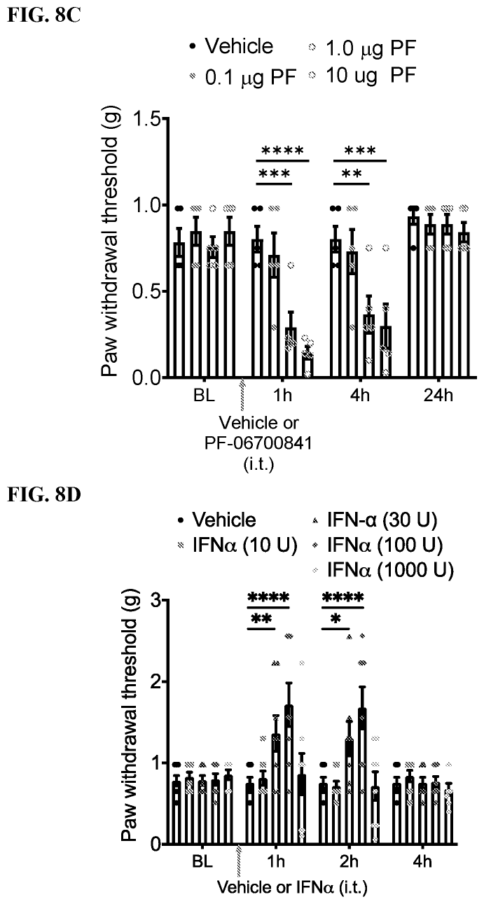

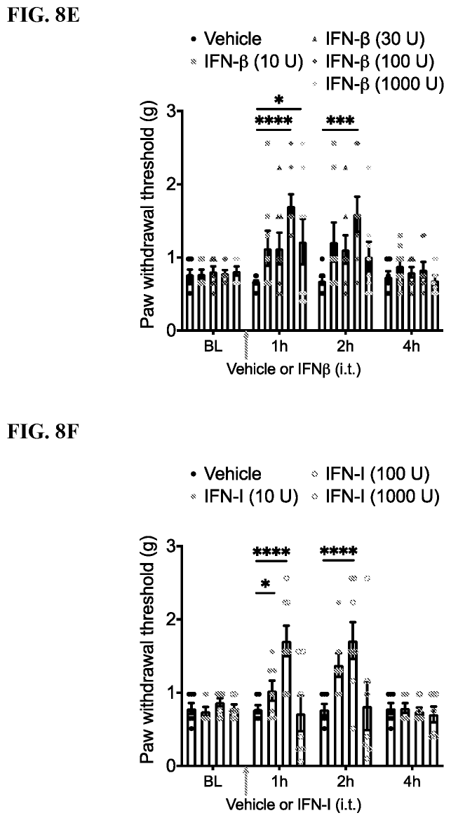

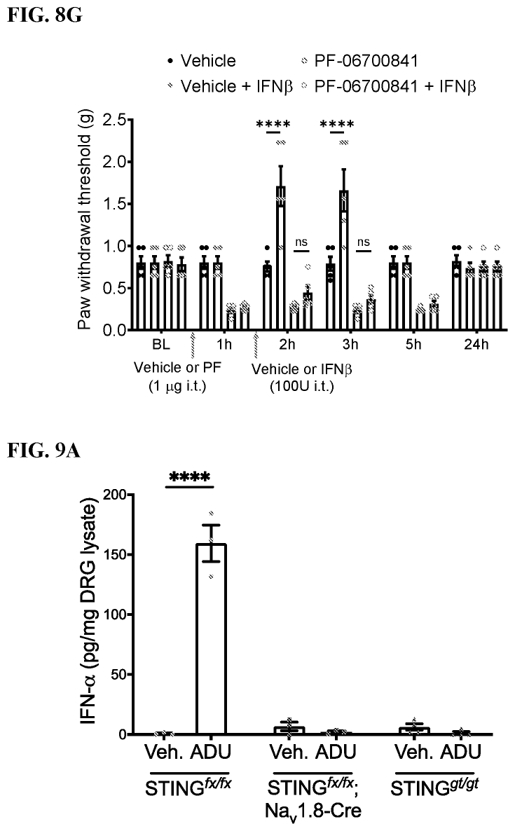

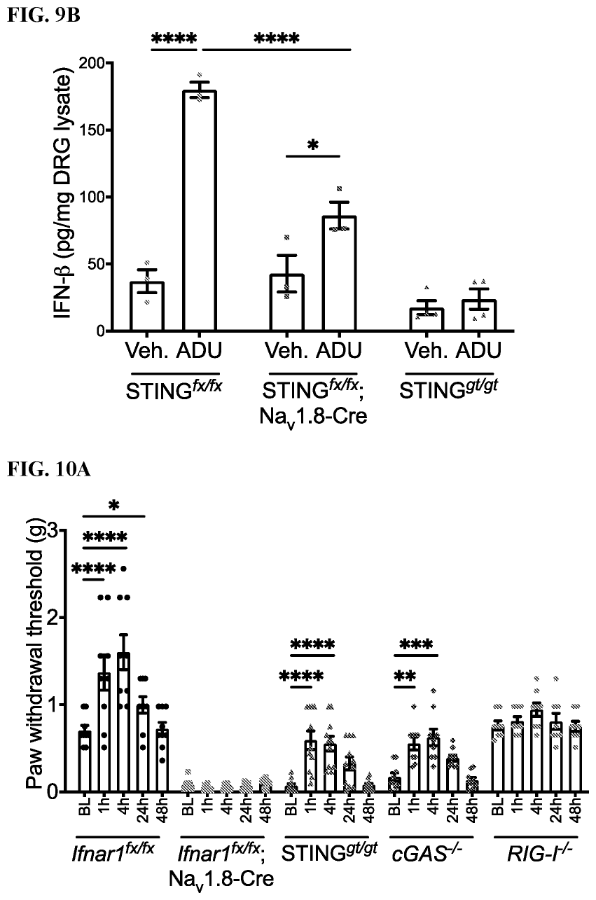

STING regulates nociception via IFN-I signaling: Activation of STING drives the production of cytokines and chemokines, with IFN-I family members (IFN-α and IFN-β) chiefly among them. Treatment with STING agonists led to a robust increase of IFN-α in serum and DRG tissue, which was abolished in STINGgt/gt mice (FIG. 2A, FIG. 7A). We also observed high basal production of IFN-β in DRG tissue, which was reduced in STING-gKO mice (FIG. 7B). In addition, treatment of cultured DRGs from STING-WT with ADU-S100 resulted in increased IFN-α and IFN-β in the culture medium (Extended Data FIG. 6c-d of Donnelly, supra). To determine which cell types within the DRG produce IFN-I in response to STING agonists in vivo, we utilized Ifnb1YFP/YFP reporter mice (Extended Data FIG. 6d of Donnelly, supra). Notably, we observed basal YFP (IFN-β) expression in DRG neurons, and this was increased after ADU-S100 treatment (Extended Data FIG. 6e-h of Donnelly, supra). Using in situ hybridization, we observed exceptionally high expression of the IFN-I receptor component Ifnar1 in virtually all DRG neurons (FIG. 2b of Donnelly, supra), which is confirmed by RNA-seq studies. Similar to STING-KO/cKO mice, Ifnar1−/− (Ifnar1-gKO) mice and mice lacking Ifnar1 selectively in sensory neurons (Ifnar1fx/fx; Nav1.8-Cre; Ifnar1-cKO) exhibited robust hypersensitivity to mechanical and cold stimuli, and CPA could be induced in Ifnar1-gKO with repeated stimulation using a sub-threshold filament (0.04 g) (Extended Data FIG. 6i-m of Donnelly, supra). Similarly, patch clamp recordings of DRG nociceptors revealed increased action potential firing rate and decreased rheobase in Ifnar1-gKO mice compared to WT littermates (Extended Data FIG. 6n-q of Donnelly, supra). Nociceptors from both Ifnar1−/− and STINGgt/gt mice exhibited increased input resistance (Extended Data FIG. 6r-s of Donnelly, supra), consistent with heightened intrinsic excitability. We also confirmed peripheral and central innervation patterns, DRG neuron numbers, and other sensorimotor behaviors were normal in Ifnar1-gKO mice (Extended Data FIG. 7a-n of Donnelly, supra). Additionally, injection of naïve mice with IFN-α or IFN-β neutralizing antibodies could recapitulate the hypersensitivity phenotype observed in Ifnar1-gKO/cK0 mice, and naïve mice treated with a pharmacological inhibitor of the Ifnar1 signaling adapter, Tyk2 (PF-06700841, i.t.) exhibited pain hypersensitivity in a dose-dependent manner (FIG. 8A-C). We also examined IFN-I levels and its function in DRGs (FIG. 9A-B). Whole-mount patch clamp recordings of DRGs revealed that incubation with anti-IFN-β (300 ng/ml, 2 h) increased action potential firing of DRG nociceptors (Extended Data FIG. 9d-e of Donnelly, supra), providing further evidence that tonic IFN-I signaling in the DRG regulates physiological nociception.

Notably, the effects of STING agonists were completely abolished at all timepoints in Ifnar1-cKO mice (FIG. 2C). In addition, we performed whole-mount patch clamp recordings of DRG nociceptors from PTX-treated Ifnar1-WT or Ifnar1-KO mice after ex vivo incubation with vehicle or ADU-S100 (FIG. 2d of Donnelly, supra). Strikingly, ADU-S100 dramatically reduced action potential firing in WT mice, and this effect was abolished in Ifnar1-deficient neurons (FIG. 2e of Donnelly, supra). Using the same incubation method in STING-WT, STING-gKO, and STING-cKO mice, we confirmed that ex vivo incubation of DRGs with ADU-S100 led to a significant increase in IFN-α and IFN-β, which was abolished in STING-KO mice and largely abolished in STING-cKO mice (FIG. 9A-B). To test whether i.t. administration of IFN-Is can produce antinociception, we injected naïve mice with recombinant IFN-a, IFN-β, or recombinant IFN-I (henceforth referred to as rIFN-I), an IFNAR agonist with cross-species activity. We observed that all three induced transient, dose-dependent antinociception (FIG. 8D-F) which corresponded to the short half-life of recombinant IFN-I proteins. Importantly, this effect was entirely dependent on Ifnar1 in peripheral sensory neurons (FIG. 2C-D). Using pharmacologic inhibitors to block distinct downstream IFN-I signaling pathways (Tyk2, PI3K, and MAPK), we found that only Tyk2 inhibition could abolish antinociception induced by i.t. IFN-β (FIG. 8G). Given that a recent study demonstrated that high concentration of IFN-α/β (300 U) can produce mechanical hyperalgesia when administered peripherally (e.g., intraplantar, i.pl.) 22, we tested whether different routes and/or concentrations of IFN-α can differentially regulate pain. Intraplantar administration of a high dose of IFN-α (300 U) led to robust mechanical hypersensitivity for up to 3 d following injection, although we observed these effects in both Ifnar1+/+ and Ifnar1−/− mice (Extended Data FIG. 8k-l of Donnelly, supra). Interestingly, we also found that i.t.

IFN-α could block the mechanical hypersensitivity induced by intraplantar IFN-α (Extended Data FIG. 8m of Donnelly, supra), suggesting that the activation of IFN-I signaling at the DRG/spinal cord levels is sufficient to produce IFN-I-mediated antinociception.

We also examined the role of STING/IFN-I signaling in cell types within the spinal dorsal horn (SDH). First, we measured the frequency and amplitude of mEPSCs from outer lamina II (IIo) spinal cord interneurons, which form a nociceptive circuit with input from C-fiber afferents and output to projection neurons. Interestingly, Ifnar1−/− mice exhibited increased frequency and amplitude of mEPSCs compared to Ifnar1+/+ littermates, while Ifnar1-cKO mice exhibited no significant differences (Extended Data. FIG. 9f-h). Examination of STING expression in the SDH revealed that STING was primarily localized to Iba1+ microglia (Extended Data FIG. 9i-j of Donnelly, supra), and STING agonists increased production of IFN-α in the SDH. IFN-β was not increased, although a relatively large amount of basal IFN-β was detected in WT mice, which was reduced in STINGgt/gt mice (Extended Data FIG. 9k-l of Donnelly, supra). Incubation of spinal cord slices with ADU-S100 decreased both the frequency and amplitude of spontaneous EPSCs (sEPSCs) from IIo interneurons (Extended Data FIG. 9m-o of Donnelly, supra), suggesting STING agonists can also attenuate neurotransmission in the spinal cord in the absence of DRG cell bodies. In addition, acute perfusion with rIFN-I reduced the frequency and amplitude of mEPSCs in spinal cord slices from WT, but not Ifnar1-gKO mice (Extended Data FIG. 9p-r of Donnelly, supra). Thus, STING-mediated IFN-I signaling in the spinal cord also plays a role in attenuating nociceptive neurotransmission.

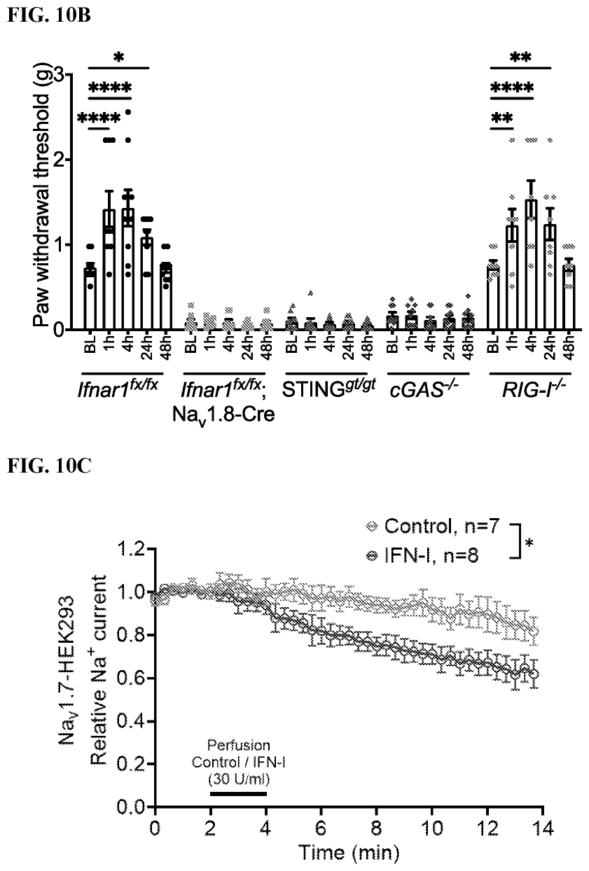

Given our observation that STING-deficient mice exhibit lower basal levels of IFN-α/β in DRG and SDH tissue, we tested whether IFN-I ligands could rescue the hypersensitivity phenotype of STINGgt/gt mice. Accordingly, we observed that i.t. IFN-α and IFN-β treatment of STINGgt/gt mice could transiently rescue mechanical hypersensitivity (Extended Data FIG. 10a of Donnelly, supra). Given that STING is activated by double-stranded DNA (dsDNA) through a mechanism involving the cytoplasmic DNA sensor cGAS, which generates 2′3′-cGAMP in response to dsDNA stimulation, we hypothesized that intracellular dsDNA may also produce antinociception through activation of the cGAS/STING pathway. Furthermore, given that IFN-I induction is a feature of both DNA sensors and RNA-sensors (such as RIG-I), we also posited that intracellular RNA may produce antinociception through a STING-independent but RIG-I/IFN-I-dependent mechanism. To test these hypotheses, we activated the intracellular dsDNA or dsRNA-sensing machinery (Extended Data FIG. 10b of Donnelly, supra) in naïve Ifnar1-WT, Ifnar1-cKO, STING-gKO, cGAS−/−, or RIG-I−/− mice via i.t. injection. The dsDNA analog poly(dA:dT) resulted in antinociception, which was abolished in Ifnar1-cKO, STING-gKO, and cGAS-KO mice, each of which also exhibited baseline mechanical hypersensitivity (FIG. 10C). In contrast, the dsRNA analog Poly(I:C) led to antinociception with similar kinetics in WT, STING-gKO, and cGAS-KO mice, but these effects were abolished in Ifnar1-cKO and RIG-I-KO mice (FIG. 10B). Thus, intracellular dsDNA and dsRNA produce IFN-I-dependent antinociception, but only dsDNA requires the cGAS/STING pathway.

To understand the mechanism underlying the antinociceptive effects of IFN-Is, we performed patch clamp recordings on DRG nociceptors. Acute perfusion with rIFN-I rapidly suppressed action potential firing, which was abolished in Ifnar1-deficient mice (FIG. 2E), and increased rheobase (FIG. 2j-k of Donnelly, supra). Given that sodium channels are critical for the generation of action potentials and physiological and pathological pain, we posited that these effects may be due to IFN-I regulation of sodium currents. Interestingly, IFN-I perfusion led to a marked reduction in sodium currents, which was abolished in Ifnar1-gKO mice, which also exhibited increased baseline sodium currents (Extended Data FIG. 10e-g of Donnelly, supra). To determine whether rIFN-I treatment could regulate activity of Nav1.7, a sodium channel critical for physiological and pathological nociception, we recorded sodium currents from HEK-293 cells stably expressing Nav1.7 following acute perfusion with rIFN-I, which led to ˜20% reduction in Nav1.7 currents (FIG. 10C). Given that calcium currents are also critical contributors to nociception, we also recorded calcium currents. Strikingly, we observed a dramatic reduction in calcium currents following rIFN-I perfusion (˜50% inhibition; FIG. 2l-m of Donnelly, supra). This reduction was abolished in Ifnar1-gKO mice, which exhibited no change in basal calcium currents (FIG. 2m-n of Donnelly, supra). Thus, IFN-I signaling can directly suppress nociceptor excitability via multifaceted suppression of sodium and calcium channel activity.

STING agonists produce analgesia in NHPs: To challenge the translational relevance of our findings, we tested whether STING activation can suppress nociception in non-human primates (NHPs), rhesus macaques (Macaca mulatta) using a menthol gel-induced cold allodynia assay. As expected based on the new results discussed above, ADU-S100 administration via a spinal catheter was accompanied by a substantial increase in cerebrospinal fluid (CSF) levels of IFN-β (FIG. 3A), as well as long-lasting and dose-dependent analgesia (FIG. 3B). These effects were observed at doses much lower than those used in murine models (3 nmol vs. 35 nmol), indicating ADU-S100 may possess better selectivity for primate/human STING. We next recorded action potentials from dissociated NHP DRG nociceptors and found that acute rIFN-I perfusion robustly suppressed action potential firing and increased rheobase (FIG. 3C-D). Finally, we tested whether rIFN-I treatment could alter the excitability of human DRG neurons (hDRGs). Although we were unable to record action potentials in hDRGs, rIFN-I perfusion consistently hyperpolarized small-diameter hDRG neurons (FIG. 3E-F). Collectively, these data reveal that STING pathway activation induces long-lasting analgesia in NHPs, and IFN-I ligands directly suppress excitability of mouse, NHP, and human nociceptors through modulation of sodium and calcium channel function (FIG. 3j of Donnelly, supra).

Our study identifies the STING/IFN-I signaling axis as a master regulator of nociception which regulates steady-state pain sensitivity as well as chronic pain in several pathological conditions. From an evolutionary perspective, the use of the STING/IFN-I signaling pathway to inhibit pain while concurrently activating the immune response following a challenge is noteworthy, as the stimuli leading to the activation of the IFN-I pathway (e.g., radiation, cancer, infection) tend to be quite painful. Thus, activation of the IFN-I pathway in sensory neurons may be a hardwired mechanism to suppress pain in challenging conditions. Our data indicate that STING-mediated IFN-I signaling acts through an autocrine and/or paracrine mechanism in peripheral sensory neurons, which acts via a noncanonical mechanism in which IFN-I/IFNAR signaling leads to exceptionally rapid suppression of sodium channel and calcium channel function (FIG. 3j of Donnelly, supra). Importantly, the intrathecal doses of STING agonists required to produce analgesia in NHPs are quite low compared to that of morphine (3 vs. 100 nmol), with effects persisting for much longer. Thus, STING agonists, at low concentrations, appear to offer significant advantages to even the most aggressive pain pharmacotherapies without showing signs of tolerance with repeated injections. Despite these promising results, it is important to consider which chronic pain conditions are severe enough to warrant immunomodulation, which could also yield unwanted side effects. The majority of patients with advanced-stage cancers experience severe pain, but fewer than half of these patients report effective pain relief. Our observation that STING agonists provide substantial antinociception in an exceptionally painful model of bone cancer pain warrant immediate further exploration. This is compounded by the clinical promise that STING agonists have shown as cancer immunotherapy adjuvants, promoting immune cell-mediated antitumor immunity. Taken further, we propose that these STING-dependent functions (e.g., antitumor immunity and antinociception) could act synergistically through distinct actions on immune cells and peripheral nociceptors.

### Example 3. Materials and Methods for Study of Cancer Pain

Reagents. The following reagents were used in this study: DMXAA (Cayman Chemical, 14617), ADU-S100 (Chemietek, CT-ADUS100), 3′3′-cGAMP (Invivogen, tlr-nacga), poly(dA:dT)/LyoVec (Invivogen, tlr1-patc), Zoledronic acid (Cayman Chemical, 14984), mouse RANKL protein (R&D systems, 462-TEC), mouse M-CSF (R&D systems, 416-ML), anti-mouse IFN-α neutralizing antibody (PBL Assay Science, 32100-1), anti-mouse IFN-β neutralizing antibody (PBL Assay Science, 32400-1) and rabbit polyclonal IgG control (Biolegend, CTL-4112).

Animals. Adult C57BL/6J mice (males and females, 8-10 weeks) were used for all behavioral and biochemical studies. STING “goldenticket” knockout mice (Stock No: 017537), Ifnar1 global knockout mice (Stock No: 028288), Rag1 knockout mice (Stock No: 002216), Ifnar1 conditional knockout mice (Ifnar1^{fx/fx.}; Stock No: 028256) and Batf3^{-/-} mice (Stock No: 013755) were purchased from the Jackson Laboratory and maintained on a C57BL/6J background. Naᵥ1.8-Cre mice, also maintained on a C57BL/6J background, were a gift from Rohini Kuner (University of Heidelberg). Behavioral testing was performed comparing STING^{gt/gt} and Ifnar1^{-/-} mice with wildtype littermate controls. These mice were maintained at an AAALAC-approved Duke University facility with two to five mice housed in each cage maintained in a 12 h light-dark cycle with ad libitum access to food and water. Animals were randomly assigned to different experimental groups. Our previous studies using similar types of behavioral and biochemical analyses were used to determine sample size. Males and females were used in a sex and age-matched manner, if not otherwise specified in the figure legends. All mouse procedures were approved by the Institutional Animal Care & Use Committee (IACUC) of Duke University. Animal experiments were conducted in strict accordance with the National Institutes of Health Guide for the Care and Use of Laboratory Animals.

Cell culture. Murine Lewis lung carcinoma cell line LL/2 (LLC1) (ATCC® CRL-1642), luciferase expressing cell line LL/2-Luc2 (ATCC® CRL-1642-LUC2™) and murine monocyte/macrophage cell line RAW 264.7 (ATCC® TIB-71) were obtained from ATCC. The mouse E0771 breast cancer cell line (94A001) was obtained from CH3 BioSystems. Cells were cultured in high glucose (4.5 g/L) Dulbecco's modified Eagle medium (Gibco, Thermo Fisher Scientific), supplemented with 10% fetal bovine serum (Gibco, Thermo Fisher Scientific) and 1% antibiotic-antimycotic solution (Sigma-Aldrich). These cells were then cultured in the presence of 5% CO2 at 37° C. Blasticidin (2 μg/ml, Gibco, Thermo Fisher Scientific) was added into LL/2-Luc2 culture medium and removed 3 days before the inoculation of mice. No testing was performed for mycoplasma contamination.

Bone cancer pain model. The murine cell lines LLC1, LL/2-Luc2 or E0771 were lightly digested using 0.05% trypsin, followed by centrifugation to remove poorly digested cell clusters. Cells were then resuspended in PBS at a concentration of 1×10⁸ cells/ml. The inoculation was performed as previously described (Wang, K. et al. “PD-1 blockade inhibits osteoclast formation and murine bone cancer pain.” The Journal of clinical investigation, (2020); Wang, Z. et al. “Anti-PD-1 treatment impairs opioid antinociception in rodents and nonhuman primates.” Sci Transl Med 12 (2020)). Briefly, mice were anesthetized with 4% isoflurane and the left leg was shaved and the skin disinfected with 10% povidone-iodine and 75% ethanol. A superficial incision (0.5-1 cm) was made near the knee joint, exposing the patellar ligament. A new 25-gauge needle was inserted at the site of the intercondylar notch of the left femur into the femoral cavity, which was then replaced with a 10 μL microinjection syringe containing a 2 μL suspension of tumor cells (2×10⁵) followed by 2 μL absorbable gelatin sponge solution to seal the injection site. The syringe contents were slowly injected into the femoral cavity over a 2-minute interval. To prevent further leakage of tumor cells outside of the bone cavity, the outer injection site was sealed with silicone adhesive (Kwik-Sil, World Precision Instruments, US). Animals with surgery related movement dysfunction or with outside bone tumor injection were excluded from the study.

Drug treatment. DMXAA was dissolved in sterile PBS containing 0.75% NaHCO₃ and ADU-S100 was dissolved in sterile PBS into 20 mg/ml, and they were further diluted 10-fold in sterile PBS prior to injection for in vivo experiments. All other reagents were dissolved in sterile saline or PBS. For experiments utilizing rabbit anti-IFN-α or -β neutralizing antibodies, a polyclonal rabbit IgG antibody served as the control. For single i.p. injection, drugs were injected on d11 after tumor inoculation. For twice i.p. injection, drugs were delivered on d3 and d7 or d10 after LLC implantation, as detailed in the results and/or figure legends.

Behavior tests. All the behavioral tests were conducted in a blinded manner and performed during between the hours of 9:00-16:00. Animals were habituated in a light and humidity controlled testing environment for at least 2 days prior to baseline testing. For Von Frey testing, mice were confined to individual 5×5 cm boxes placed on an elevated wire grid. A blinded experimenter stimulated their hindpaws using a series of von Frey filaments with logarithmically increasing stiffness (0.02-2.56 g, Stoelting). Each filament was applied perpendicularly to the central plantar surface. The 50% paw withdrawal threshold was determined using Dixon's up-down method. In addition, we also measured paw withdrawal frequency to repeated stimulation (10 times, with ˜1-2 minutes between each stimulation) using a subthreshold 0.16 g von Frey filament, which is a more sensitive method to detect mechanical allodynia. To measure cold allodynia, mice were similarly isolated to individual boxes on an elevated mesh floor, and a drop (˜20-30 μl) of acetone was applied to the plantar hindpaw. The duration of time that animal displayed a nociceptive response (lifting or licking the paw) over a 90 s period immediately after acetone application was recorded. To measure locomotor function, we performed open field testing in which mice were placed in the center of a 45×45 cm chamber and locomotor activity was recorded by an overhead webcam connected to a laptop computer, and animals' movements were automatically tracked for 30 minutes using ANY-Maze. The total distance traveled and mean speed during the 30-minute period were analyzed.

In vivo X-ray radiography. Osteolytic bone destruction was continuously evaluated by radiography using the MultiFocus by Faxitron system (Faxitron Bioptics LLC, Tucson, Ariz.). Radiographs of tumor-bearing femora were rated for bone destruction on a 0-5 score scale based on previous study: 0 for normal bone at baseline without tumor inoculation; 1 for one to three radiolucent lesions indicative of bone destruction compared to baseline; 2 for increased number of lesions (three to six lesions) and loss of medullary bone; 3 for loss of medullary bone and erosion of cortical bone; 4 for full-thickness unicortical bone loss; 5 for full-thickness bicortical bone loss and displaced skeletal fracture. All radiographic image quantifications were completed by an experimenter who was blinded to the experimental conditions.

Microcomputed tomography. Microcomputed tomography (MicroCT) analyses were performed on femurs from tumor inoculated mice or naïve mice using a VivaCT 80 scanner with the 55-kVp source (Scanco, Southeastern, Pa.) as previously described (Wang, C. et al. “NOTCH signaling in skeletal progenitors is critical for fracture repair.” The Journal of Clinical Investigation 126, 1471-1481 (2016); Cao, C. et al. “Increased Ca2+ signaling through CaV1.2 promotes bone formation and prevents estrogen deficiency-induced bone loss.” JCI Insight 2, (2017)). Quantification of microCT data was calculated for distal femurs of mice treated with vehicle or DMXAA. Parameters quantified included bone volume/total volume (BV/TV) and connectivity density (Conn.D) within a region of 100 slides and 200 slides proximal to the distal growth plate.

Bone histology, TRAP, and ALP staining on mouse femurs. Mice were deeply anesthetized and perfused intracardially with 4% paraformaldehyde (PFA) in 0.1M phosphate buffered saline. The femora were removed and then post-fixed for 48 h in the same fixative at 4° C. After demineralization in EDTA (10%) for 10 days, femur samples were dehydrated in an ascending gradient of ethanol (30-100%) followed by paraffin embedding. Serial sections for trabecular bone were obtained from the distal femur at a thickness of 5 μm followed by tartrate-resistant acid phosphatase (TRAP) staining or alkaline phosphatase (ALP) staining using TRAP kits (Fast Red TR/Naphthol AS-MX, Sigma, St. Louis, Mo.) and NBT/BCIP (Thermo Scientific), respectively. Bone static histomorphometric analyses for osteoclast number (osteoclast number per trabecular bone surface covered by osteoclasts, Oc.S/BS) and osteoblast number (osteoblast number per trabecular bone surface, Ob.N/BS) were conducted using Image J (NIH) based on images taken by a Leica Q500MC microscope. Osteoclasts, osteoblasts and trabecular bone at the metaphysis of the femur (1500 μm proximal to the distal growth plate) were quantified, since bone destruction in this model mainly occurs in this area. Three sections per animal were randomly chosen and used for quantification.

Visual and immunohistochemical analysis of lung metastases. Mice were deeply anesthetized with isoflurane and perfused intracardially with PBS, followed by 4% PFA. After the perfusion, lungs were removed from mice and post-fixed in the same fixative overnight. The samples were then dehydrated with a 30% sucrose solution, embedded in O.C.T. (Tissue Tek), and cryosectioned to produce 8 μm thick sections. For Hematoxylin and Eosin (H & E) staining, lung sections were rehydrated and stained with 0.1% Hematoxylin and 0.5% Eosin in sequence. After dehydration and clearance with HistoClear (Electron

Microscopy Sciences, Hatfield, Pa.), slides were mounted with a resinous mounting medium (Mercedes Medical, Sarasota, Fla.) and subsequently imaged using a Leica Q500MC microscope with a digital camera at different magnifications.

Bone marrow collection. Mice were humanely euthanized and femora were carefully removed and placed on ice. The muscles surrounding the femur were gently removed as much as possible, followed by separation of the distal epiphysis from femoral shaft. The proximal end of the femur removed, followed by insertion of a 25-gauge needle into the distal end of the femur and injection of 1 ml cold PBS or α-MEM (Gibco, Thermo Fisher Scientific) by lightly pressing down on the plunger, allowing the bone marrow to be evacuated into a 1.5 ml centrifuge tube. Bone marrow was harvested through centrifugation at 800 g for 5 minutes at 4° C. for subsequent detections or cell cultures.

ELISA. Mouse high-sensitivity IFN-α ELISA kit (42115-1) and IFN-β ELISA kit (42410-1) were purchased from PBL Assay Science. Mouse CTX-I ELISA kit (AC-06F1) and mouse PINP ELISA kit (AC-33F1) were purchased from Immunodiagnostic Systems. ELISA was performed using culture medium, serum, and bone marrow lysates. Serum was obtained from whole blood that was collected from a submandibular vein via facial vein puncture, coagulated for 30 minutes at room temperature, followed by centrifugation (2,000 ×g for 10 min, 4° C.) and collection of the supernatant (serum). Bone marrow was homogenized in a lysis buffer containing protease inhibitors at 4° C. for 30 minutes. ELISA was conducted in accordance with the manufacturer's instructions. A standard curve was performed for each experiment.

In vitro induction of osteoclastogenesis and TRAP staining. For in vitro drug treatment, the RAW 264.7 cells were incubated with 35 ng/ml RANKL for six days. Isolated bone marrow cells were cultured overnight in α-MEM media containing 10% fetal bovine serum and 1% antibiotic-antimycotic solution. The suspended cells were collected and incubated with 20 ng/ml MCSF for 3 days to obtain bone marrow-derived macrophage (BMDM). The attached cells were further activated by 35 ng/ml RANKL and 20 ng/ml MCSF. Drug treatment was performed in tandem with RANKL. Culture medium and co-cultured reagents were changed every 3 days. After 6 or 7 days of incubation, the cells were fixed by 4% PFA and stained with warm TRAP staining solution (TRAP kit, Sigma-Aldrich, SLBW4002) for 10-30 min at 37° C. TRAP-positive multinucleated cells that displayed three or more nuclei under a light microscope were considered osteoclasts, and the numbers of positive cells were counted in a blinded fashion with images of randomly selected visual fields (4-5 regions per well) using Image J software.

Flow cytometry. For analysis of immune subsets present within the bone marrow tumor microenvironment, bone marrow was collected as previously described followed by removal of RBC cells using RBC lysis buffer (Sigma, R7757). Cells were subsequently washed with PBS and resuspended in 2% paraformaldehyde in PBS for 10 minutes. Fixed cells were washed several times with PBS, followed by incubation in blocking buffer (1% anti-mouse-CD16/CD32, 2.4 G2, 2% FBS, 5% NRS, 1% triton ×100 and 2% NMS in HBSS; BD Bioscience) for 1 h at room temperature. Cells were subsequently stained with IL-17-FITC (1:20, rat, Miltenyi Biotec, 130-102-262), CD-3 APC/cy7 (1:200, rat, Biolegend, 100221), CD-4 APC (1:200, rat, Biolegend, 100411), FoxP3-PE (1:20, human, Miltenyl Biotec, 130-111-678), CD8a-FITC (1:200, rat, Biolegend, 100705), and CD11b-PE(1:200, rat, Biolegend, 101207) in blocking buffer for 1 h at room temperature. After staining, cells were washed in PBS with EDTA. Flow cytometry events were acquired by a BD FACS Canto II flow cytometer using the BD FACS Diva 8 software (BD Bioscience). Data were analyzed using Cytobank software (https://www.cytobank.org/cytobank).

In vivo bioluminescence imaging. RediJect D-Luciferin Ultra was purchased from PerkinElmer (770505). Prior to in vivo imaging, mice were shaved in the region of interest depicted in the figure. Bioluminescence images of LL/2-Luc2 bearing mice were captured with IVIS Lumina III system 15 min after intraperitoneal injection of D-Luciferin (30 mg/kg). The IVIS acquisition control panel was set to following conditions for imaging: Exposure time=auto, Binning=medium, F/Stop=1, Emission Filter=open. The bioluminescence images were analyzed using Living Image software from PerkinElmer.

Whole-cell patch clamp recordings in whole-mount DRGs ex vivo. Four-week-old male C57BL/6 mice were used to establish the bone cancer pain model by intrafemoral inoculation of LLC, leading to nociceptor hyperexcitability in the ipsilateral L3-L5 DRGs which extend afferent nerve fibers to the tumor-bearing femur. Notably, young mice were used for these experiments due to technical limitations in performing electrophysiological recordings on older mice. 11d after tumor implantation, mice were euthanized followed by careful isolation of L3-L5 DRGs, which were placed in oxygenated artificial cerebrospinal fluid. DRGs were lightly digested for 20 minutes using an enzymatic mixture consisting of 0.32 ml collagenase A (1 mg/mL) and Trypsin (0.25%). Intact DRGs were then incubated in ACSF oxygenated with 95% O2 and 5% CO2, supplemented with vehicle (PBS) or 30 μM DMXAA in PBS for 2 hours at 37° C. Following incubation, DRGs were transferred to a recording chamber, where neurons could be visualized using a 40× water-immersion objective on an Olympus BX51WI microscope. Patch pipettes were pulled from borosilicate capillaries (Chase Scientific Glass Inc.) and filled with a pipette solution containing (in mM): 126 potassium gluconate, 10 NaCl, 1 MgCl2, 10 EGTA, 2 Na-ATP, and 0.1 Mg-GTP, adjusted to pH 7.3 with KOH. The external solution was composed of (in mM): 140 NaCl, 5 KCl, 2 CaCl2, 1 MgCl2, 10 HEPES, 10 glucose, adjusted to pH 7.4 with NaOH. The resistance of pipettes was 4-5 MΩ Series resistance was compensated for (>80%) and leak subtraction was performed. Data were low-pass filtered at 2 KHz and sampled at 10 KHz. Data were recorded and analyzed using the pClamp10 (Axon Instruments) software.

Data analysis and statistics. The sample sizes for each experiment were based on our previous studies on such experiments. Statistical analysis was performed with GraphPad Prism 6.0 (GraphPad Software). All the data in the figures are expressed as mean±standard error (SEM). Biochemical and behavioral data were analyzed using a two-tailed t-test (two groups), One-Way ANOVA, or Two-Way ANOVA, followed by post-hoc Bonferroni test. Fisher's exact test was utilized for the comparison of the bone fracture ratio. The criterion of P<0.05 was defined as the threshold for statistical significance. In each figure, significance denotes P values as follows: * P<0.05; ** P<0.01; *** P<0.001, **** P<0.0001.

Study approval. The present studies in animals were reviewed and approved by the IACUCs of Duke University. All animal procedures were conducted in accordance with the NIH's Guide for the Care and Use of Laboratory Animals (National Academies Press, 2011)

### Example 4. STING Suppresses Cancer Pain via Immune and Neuronal Modulation

Patients with advanced lung, breast, thyroid and bladder cancers frequently suffer from cancer pain following bone metastasis, which is accompanied by osteolytic lesions and severe pain. Studies estimate that approximately 75% of patients with late stage cancer experience moderate or severe pain, and more than half of all patients with metastatic cancer pain report insufficient pain relief by the currently available pharmacotherapies. Inadequate pain control for patients with metastatic cancer is often accompanied by depression, anxiety, impaired function, and significantly reduced quality of life, leading to increased morbidity and mortality. Thus, in addition to the ongoing challenge of developing new therapeutics capable of treating the underlying cancer, there is also an urgent unmet clinical need to develop new therapies which provide cancer patients with palliative care to relieve pain and improve quality of life. Notably, endocannabinoid has been shown to alleviate murine bone cancer pain without producing the side effects of opioids. An ideal therapeutic approach would be one that is capable of actively treating the underlying cancer while concurrently suppressing cancer-associated pain, thereby treating the pain and the underlying disease.

STING, or stimulator of interferon (IFN) genes, is an intracellular DNA sensor which plays a critical role in innate immunity, promoting the elimination of pathogens and damaged host cells via the induction of type-I IFN (IFN-I), including IFN-α and IFN-β Activation of the STING pathway can also potently enhance antitumor immunity, underscored by preclinical studies in which the murine STING agonist DMXAA or the cross-species STING agonist ADU-S100 have been demonstrated to suppress tumor progression and increase survival in an adaptive immune cell-dependent manner. In addition, several groups have demonstrated STING-activating- micro- or nanoparticles also show efficacy in promoting innate and adaptive immunity in orthotopic and genetically engineered tumor models in mice. These studies have led to the exploration of ADU-S100 and other small molecule STING agonists to be tested as potential immunotherapy agents in several ongoing clinical trials. However, it remains unclear if STING agonists are effective in treating metastatic bone cancer, especially given that bone marrow is regarded to be an immunosuppressive tumor microenvironment.

Previous studies have demonstrated that IFN-I signaling suppresses osteoclast formation, and activation of the STING pathway promotes bone formation in a murine bone autoimmune disease model. This is noteworthy, as tumor-induced over-activation of osteoclasts is a dominant mechanism leading to osteolytic bone lesions and cancer pain. It is generally believed that activation of pain-sensing nociceptive neurons (nociceptors) by soluble mediators released from cancer cells and osteoclasts drives bone cancer pain. Based on these studies, and taken in conjunction with the promise STING agonists have shown as cancer immunotherapy agents, we posited that activation of the STING pathway in metastatic bone cancer may be a unique synergistic approach to concurrently promote antitumor immunity, suppress bone destruction, and provide pain control. In this study, we tested this hypothesis using syngeneic mouse models of bone cancer pain in which lung or breast cancer cells are introduced into the intramedullary canal of murine femurs. Using this model, we administered multiple STING agonists and measured the effects of STING pathway activation on metastatic bone cancer-induced pain, bone destruction, and local tumor burden.

STING agonists attenuate bone cancer-induced pain and restore locomotor function. We first sought to determine whether activation of STING via systemic administration of DMXAA could provide long-term therapeutic effects in a mouse model of metastatic bone cancer. To this end, we established a syngeneic murine bone cancer pain model by inoculating Lewis lung carcinoma (LLC, 2×10⁵ cells in 2 μl) cells into the femora of C57BL/6 mice. Vehicle or DMXAA (20 mg/kg) was intraperitoneally (i.p.) injected twice to these mice on day 3 (3 d) and day 7 (7 d) after tumor implantation. Behavioral tests including von Frey testing for mechanical allodynia and acetone response duration for cold allodynia were performed on the hindpaw of the tumor-bearing leg at baseline (BL), 7 d (before drug injection), 10 d, and 14 d after LLC inoculation. Flinches and guarding behavior for spontaneous/ongoing pain was evaluated on day 14 post tumor injection (FIG. 1a of Wang et al. “STING suppresses cancer pain via immune and neuronal modulation.” bioRxiv 2021.01.18.426944). DMXAA treatment significantly reduced mechanical allodynia on d7, d10 and d14 (FIG. 1b of Wang, supra) and cold allodynia on d10 and d14 after LLC implantation (FIG. 1c of Wang, supra). On d14, DMXAA treatment also attenuated measures of spontaneous and ongoing pain (FIG. 1d of Wang, supra). No apparent sex differences were observed, as the therapeutic effect of DMXAA on bone cancer pain existed in both male and female mice (Extended Data FIG. 1a-d of Wang, supra). In addition, we observed no differences in body weight between the vehicle- and DMXAA-treated groups (FIG. 1e of Wang, supra), indicating the experimental protocol we used is relatively safe and without gross systemic gastrointestinal (GI) toxicity. Notably, survival was not the endpoint of this study and given that animals in late stages of this model experienced severe pain and functional impairment, all mice were sacrificed at d17 post-inoculation to maintain reasonable health conditions and minimize suffering, as indicated in our recent study (Wang, K. et al. “PD-1 blockade inhibits osteoclast formation and murine bone cancer pain.” The Journal of clinical investigation, (2020)).

Clinically, a critical comorbidity of bone metastasis in patients with advanced stage cancers is diminished or lost mobility, leading to functional impairment and reduced quality of life. To determine whether STING activation with DMXAA could improve locomotor function, we evaluated the movement activity in the open field test. Importantly, mice treated with DMXAA exhibited greater overall distance of movement and increased speed of movement on d14 after tumor inoculation (FIG. 1f of Wang, supra). Thus, systemic treatment with DMXAA significantly improved locomotor function in mice with bone cancer.

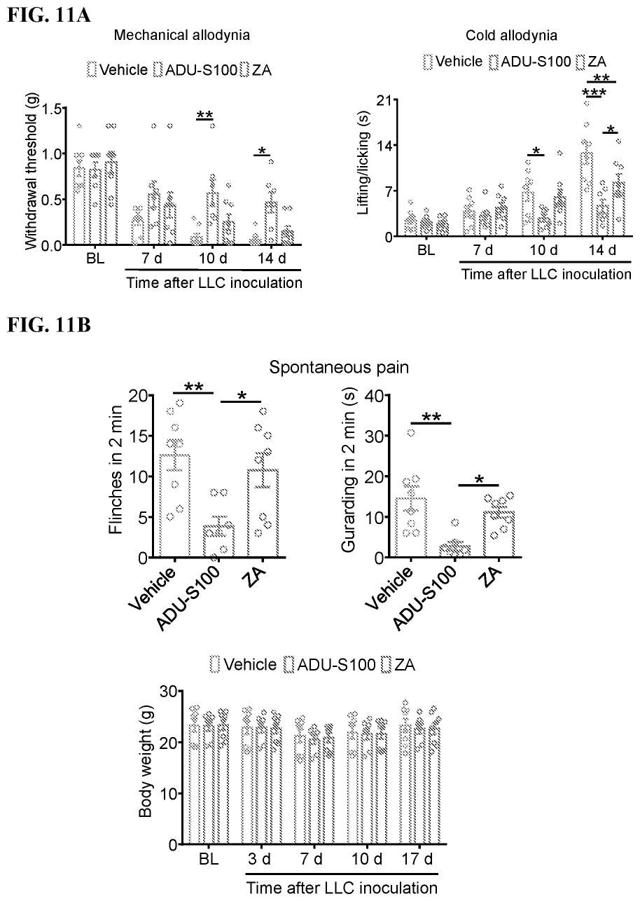

Clinically, bisphosphonates are widely used for the prevention and treatment of metastatic bone cancer-induced skeletal-related events (SREs) by promoting apoptosis of bone-resorbing osteoclasts. Zoledronic acid (ZA) is one of the most potent bisphosphonates, and has also been reported to exhibit antitumor effects. Moreover, given the limited translational significance of DMXAA due to its specificity for murine STING, we also sought to test whether ADU-S100 could exert similar therapeutic effects. Following administration of vehicle, ADU-S100 (20 mg/kg), or ZA (100 μg/kg, a highly effective dose with minimal toxicity, as demonstrated in previous studies at d3 and d10, we found that ZA failed to reduce cancer-induced mechanical allodynia when analyzing paw withdrawal threshold, but could reduce withdrawal frequency to low-threshold stimulation (0.16 g Von Frey filament) on d10 post tumor implantation. ADU-S100 treatment, however, could significantly attenuate mechanical allodynia in both measures on d10 and d14, with effects superior to those of ZA (FIG. 11A). ZA reduced cold allodynia on d14 after tumor inoculation, whereas ADU-S100 reduced cold allodynia on both d10 and d14, and this effect was significantly greater in the ADU-S100 group compared to the ZA group on d14 (FIG. 11A). In addition, mice treated with ADU-S100 but not ZA exhibited reduced spontaneous and ongoing pain compared to vehicle-treated mice (FIG. 11B). We found that these doses of ADU-S100 and ZA again had no effect on overall body weight (FIG. 11B), suggesting they are relatively safe and without gross GI toxicity. To again assess the potential benefits of ZA and ADU-S100 on function and mobility, we performed open field testing at d14 on mice treated with vehicle, ADU-S100 and ZA at d3 and d10. Notably, mice in the ADU-S100 treatment group but not the ZA treatment group exhibited increased overall distance of movement and increased speed of movement compared with the vehicle treatment group (FIG. 1k of Wang, supra). Taken together, ADU-S100 was superior to ZA in reducing cancer-induced pain and improving locomotor function.

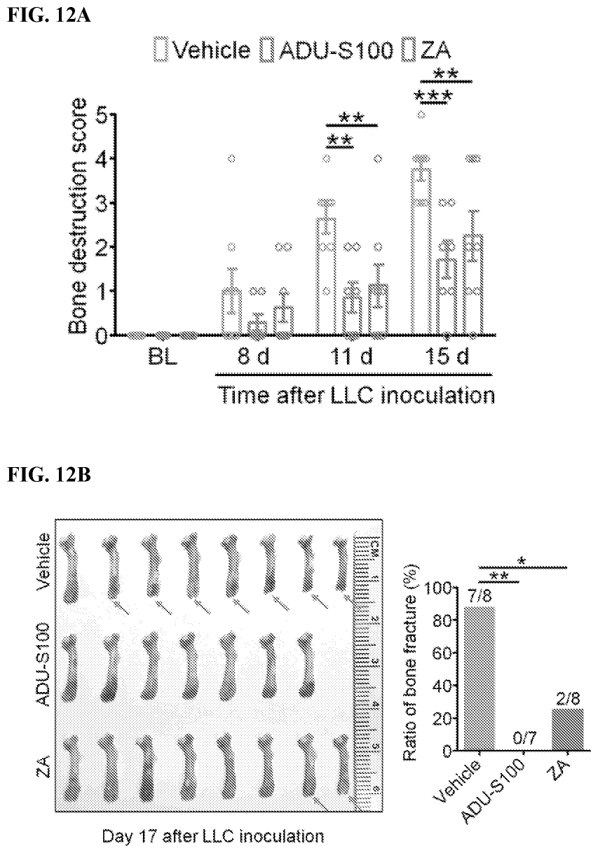

STING agonists protect against cancer-induced bone destruction. Bone cancer-induced pain usually develops in tandem with the onset of tumor-induced bone destruction. It is understood that bone cancer pain is evoked by factors produced directly by cancer cells which act on afferent nociceptive nerve fibers in the tumor microenvironment (TME). In addition, cancer cells promote bone cancer pain indirectly by accelerating osteoclastogenesis, generating osteoclasts which release pro-nociceptive factors and promote bone resorption, facilitating bone destruction and painful fractures. Thus, cancer cells in the bone tumor microenvironment promote bone cancer through direct and indirect mechanisms (Extended Data FIG. 1e. of Wang, supra). In vivo, the LLC cell line is known to induce osteolytic bone destruction due to tumor-induced activation of osteoclast formation and activity, recapitulating the pathogenesis of metastatic bone cancers in humans. Thus, we sought to continuously assess bone destruction using radiography in tumor-bearing mice treated with vehicle or DMXAA (20 mg/kg, i.p. at d3 and d7). The grade of bone destruction was scored on a range from 1 to 5 using high-resolution X-ray radiographs of tumor bearing femora, as described by Honore et al (“Osteoprotegerin blocks bone cancer-induced skeletal destruction, skeletal pain and pain-related neurochemical reorganization of the spinal cord.” Nat Med 6, 521-528 (2000)). DMXAA treatment decreased the bone destruction score on d8, d11 and d15 after LLC inoculation compared with vehicle group (FIG. 2a-b of Wang, supra). No sex differences were observed in the protective effects of DMXAA on bone destruction (Extended Data FIG. 1f of Wang, supra). To explore the microarchitecture of bone, we also employed micro computed tomography (Micro-CT) with 3-dimentional reconstruction analysis ex vivo on the distal aspect of tumor-bearing femurs. On d11 after tumor implantation and vehicle or DMXAA treatment, 3D reconstruction showed less bone cancer-induced trabecular bone loss and fewer cortical bone lesions in DMXAA-treated mice compared with vehicle group (FIG. 2c of Wang, supra). Quantitative assessments for bone microstructural parameters demonstrate there is higher trabecular bone connectivity density (Conn.D) and increased cortical bone volume/total volume (BV/TV) in mice administered DMXAA (FIG. 2d of Wang, supra). Next, we analyzed bone destruction by X-ray radiography following treatment with ADU-S100 or ZA (d3 and d10, as in FIG. 1f of Wang, supra). Similar to DMXAA, both ADU-S100 and ZA treatment reduced the bone destruction score at d11 and d15 after tumor inoculation (FIG. 12A).

Development and progression of cancer-induced osteolytic bone destruction frequently leads to bone fracture, which is an important component of SREs in patients with bone metastasis and is associated with decreased overall survival. On d17 after LLC inoculation, mice were euthanized and the tumor bearing femora were collected and the distal tumor-bearing femur where bone destruction occurs was analyzed. Notably, we found that 87.5% (7/8 mice) of vehicle-treated mice suffered bone fractures, whereas only 12.5% (1/8 mice) mice treated with DMXAA developed distal bone fractures (FIG. 2g of Wang, supra). Likewise, 0% (0/7 mice) in the ADU-S100-treated group and 25% (2/8 mice) in the ZA group developed bone fractures (FIG. 12B), indicating both STING agonists and ZA could significantly reduce bone destruction.

STING agonist treatment protects against breast cancer induced bone pain and bone destruction. Similarly to lung cancer, breast cancer is also prone to metastasize to bones and cause bone destruction. To explore the potential protective effect of STING agonists in breast cancer-induced bone destruction, we utilized the E0771 medullary breast carcinoma cell line to establish a syngeneic mouse model of breast cancer-induced bone cancer pain in female C57BL/6 mice. Similar to the LLC line, tumors established with the E0771 line also induce osteolytic bone lesions. After intra-femur inoculation, mice were treated with vehicle, DMXAA or ADU-S100 followed by behavioral testing and X-ray radiography of tumor-bearing femurs (FIG. 3a of Wang, supra). Similar to our results in the LLC bone cancer pain model, we found that DMXAA and ADU-S100 treatment could markedly reduce mechanical allodynia, cold allodynia and spontaneous pain compared to vehicle treatment (FIG. 3b-d of Wang, supra) but had no effect on body weight (FIG. 3e of Wang, supra). Furthermore, both DMXAA and ADU-S100 could also attenuate bone destruction scored from X-ray images of the E0771-bearing femora (FIG. 3f-g of Wang, supra). Thus, STING agonists can protect against cancer-induced bone pain and bone destruction caused by multiple cancer subtypes prone to bone metastasis.

Protective effect of DMXAA on bone pain and bone destruction is STING dependent. To verify the antinociceptive and bone anabolic effects of DMXAA are mediated by STING, WT mice and STING “goldenticket” knockout (STING^{gt/gt}) mice were inoculated with LLC cells intrafemorally followed by vehicle or DMXAA (20 mg/kg) administration (i.p.) on d3 and d7 post LLC injection. Notably, STING^{gt/gt} mice displayed markedly reduced hindpaw withdrawal threshold and increased withdrawal frequency in von

Frey tests compared to WT mice at baseline. DMXAA treatment significantly attenuated mechanical and cold allodynia in WT mice, and this effect was abolished in STING^{gt/gt} mice (Extended Data FIG. 2a-b of Wang, supra). We also measured cancer-induced bone destruction in these mice using radiographic examination of bone destruction of the tumor-bearing distal femurs. We observed a reduction in the bone destruction score in DMXAA-treated WT mice at d11 and d15 after tumor inoculation, and this effect was abolished in STING^{gt/gt} mice (Extended Data FIG. 2c-d of Wang, supra). We did not see body weight changes after the experimental manipulations (Extended Data FIG. 2e of Wang, supra). This shows that the protective effects of DMXAA on cancer-induced pain and bone destruction are mediated by STING.

IFN-I signaling mediates the protective effects of STING agonists in bone cancer. STING activation leads to the transcriptional induction of interferon response genes and the robust production and release of type-I interferons, including IFN-α and IFN-β. To confirm that systemic administration of STING agonists leads to increased IFN-I response both systemically and locally within the tumor microenvironment, we analyzed the level of IFN-α and IFN-β by ELISA. We found that serum levels of IFN-α increased approximately 1000-fold 4 h after a single i.p. injection of DMXAA (20 mg/kg) or ADU-S100 (20 mg/kg) on d3 after tumor inoculation compared to vehicle group, and this increase was maintained for up to 24 hours. Meanwhile, serum IFN-β levels were also dramatically upregulated 4 h after DMXAA and ADU-S100 administration (FIG. 4a of Wang, supra). On d3 after LLC implantation, the bone marrow (BM) from tumor bearing femora were also collected 4 h after vehicle, DMXAA or ADU-S100 i.p. treatment and analyzed by ELISA. Both IFN-α and IFN-β were sharply increased in BM lysate in mice treated with DMXAA or ADU-S100 (FIG. 4b of Wang, supra). Thus, systemic administration of STING agonists promoted a robust IFN-I response systemically and in the bone cancer tumor microenvironment.

The IFN-α/β receptor (IFNAR) is a heterodimeric signal transducing receptor complex composed of Ifnar1 and Ifnar2, each of which is required for IFN-I signaling. To test how IFN-I signaling contributes to the protective effects of STING agonists in the bone cancer model, we again introduced LLC cells into the femora of Ifnar1^{+/+} (WT) or Ifnar1^{-/-} (KO) mice to establish the bone cancer models in mice with deficient host IFN-I signaling. Similar to mice lacking STING, we found that Ifnar1^{-/-} mice exhibited mechanical hypersensitivity at baseline compared to WT littermate control mice (FIG. 4c of Wang, supra). Following treatment with vehicle or DMXAA (20 mg/kg, i.p. at d3 and d7), we found that DMXAA treatment effectively attenuated cancer-induced mechanical allodynia and cold allodynia in WT mice but not Ifnar1^{-/-} mice (FIG. 4c-d of Wang, supra). On d11 and d15 after tumor inoculation, DMXAA treatment also significantly reduced the bone destruction score without changing overall body weight in WT mice, but this effect was abolished in Ifnar1^{-/-} mice (FIG. 4e-g of Wang, supra). Therefore, host IFN-I signaling through Ifnar1 is required for the protective effects of STING agonists on cancer pain and bone destruction induced by bone cancer.

DMXAA inhibits bone cancer-induced hyperexcitability of DRG nociceptive neurons. Given that cancer-evoked pain in our bone cancer model is transduced by peripheral nociceptors in the dorsal root ganglion (DRG), we next sought to determine whether the antinociceptive effects of STING agonists in bone cancer pain are due to direct effects on nociceptor excitability. To this end, WT mice were inoculated with LLC cells to establish bone cancer models and lumbar L3-L5 DRGs were isolated on d11 and incubated ex vivo with vehicle or DMXAA (30 μM) for 2 h followed by patch clamp recordings to measure nociceptor excitability (FIG. 5a of Wang, supra). Importantly, compared to vehicle, DMXAA incubation of DRGs markedly increased the rheobase of nociceptors, a measure of the current required to evoke action potentials (FIG. 5b-c of Wang, supra). In addition, we found that bone cancer increased neuronal excitability and acute DMXAA incubation sharply reduced the cancer-induced increase in current-evoked action potential firing compared with vehicle-treated DRGs (FIG. 5d-e of Wang, supra). Taken together, these data indicate that STING activation with DMXAA can suppress cancer-induced hyperexcitability of DRG nociceptors, and cells present within the DRG are sufficient to mediate these effects.

Repeated administration of STING agonists may suppress cancer-induced pain by reducing tumor burden, reducing bone destruction, by a neuronal mechanism involving direct suppression of nociceptor activity, or a combination of all three of these mechanisms. Our electrophysiological data indicate that STING agonists can suppress bone cancer-induced pain via a direct neuronal mechanism which is independent to effects on tumor growth or bone destruction. To test whether acute administration of STING agonists can suppress bone cancer-induced pain, we performed behavior tests in mice on d11 after tumor inoculation 4 h after single i.p. injection of vehicle, DMXAA, or ADU-S100. Notably, both STING agonists induced a substantial reduction in mechanical allodynia, cold allodynia, and spontaneous pain (FIG. 5f-h of Wang, supra). Since the natural activators of STING are intracellular double-stranded DNA (dsDNA) and the bacterial cyclic dinucleotide 3′3′-cGAMP, we also assessed whether dsDNA and 3′3′-cGAMP could produce antinociception in the bone cancer model. Interestingly, i.p. administration of dsDNA (30 μg, complexed with LyoVec to facilitate cellular penetration) or cGAMP (20 mg/kg) could attenuate cold allodynia or/and mechanical allodynia 4 h after injection on d11 post LLC implantation (Extended Data FIG. 3a-d of Wang, supra). Additionally, we tested whether the acute antinociceptive effects of DMXAA were STING- and Ifnar1-dependent by injecting DMXAA (20 mg/kg, i.p.) into WT, STING^{gt/gt} mice and Ifnar1^{-/-} mice, measuring mechanical and cold allodynia 4 h after injection. We found DMXAA could reduce mechanical allodynia and cold allodynia in WT mice but not in STING^{gt/gt} mice or Ifnar1^{-/-} mice (FIG. 5i-j of Wang, supra). To further determine whether neuronal IFN-I signaling is responsible for the acute antinociceptive effects of STING agonists, we established the bone cancer pain model using mice lacking Ifnar1 selectively in sensory neurons (Ifnar1^{fx/fx}; Naᵥ1.8-Cre; Ifnar1-cKO), or their wildtype littermates. Importantly, we found the antinociceptive effects conferred by a single administration of DMXAA (20 mg/kg, i.p.) were present in WT, but not Ifnar1-cKO littermates (FIG. 5k-l of Wang, supra). Given the immediacy of these effects, and taken in conjunction with our electrophysiological data, we conclude that STING agonists exert antinociceptive effects via direct actions on nociceptors in an Ifnar1-dependent mechanism.

STING agonists suppress local bone cancer tumor burden and further metastasis. Intratumoral injection of STING agonists has been reported to reduce tumor growth by promoting T cell-mediated antitumor immunity in several preclinical animal studies. It is unknown, however, whether systemic administration of STING agonists can attenuate tumor progression in the bone marrow, which is generally regarded as an overwhelmingly immunosuppressive tumor microenvironment. To answer this question, luciferase-labeled LLC cells (LL/2-LUC2 cell line) were used to establish the metastatic bone cancer model via intrafemoral inoculation, thereby enabling measurement of local tumor burden by in vivo bioluminescent imaging. Mice were treated with vehicle or DMXAA (20 mg/kg, i.p. at d3 and d7), followed by in vivo bioluminescence imaging at d8, d11, and d15. Notably, mice treated with DMXAA exhibited lower local tumor burden at d11 and d15, as measured by total flux of LL/2-Luc2 cells in tumor-bearing mice (FIG. 6a of Wang, supra). By d17, tumor growth beyond the normal anatomic boundaries of the distal femur could be visually observed, leading to an increase in the circumference of the tumor-inoculated (ipsilateral) thigh compared to the contralateral side. To quantify this, we measured the ratio of the maximum thigh circumference (ipsilateral/contralateral), which accurately reflects local tumor volume. Notably, we found that DMXAA and ADU-S100 treatment, but not ZA treatment, could reduce the ratio of maximum thigh circumference compared to the vehicle-treated group on day 17 in both the LLC and E0771-induced bone cancer models (FIG. 6b-c of Wang, supra). To test whether these effects were dependent on host-intrinsic STING and Ifnar1, we measured local tumor burden using thigh circumference at d17 in STING^{gt/gt} and Ifnar1^{-/-} mice and found that this protective effect was abolished in mice lacking either STING or Ifnar1 (Extended Data FIG. 4a-b of Wang, supra). Thus, systemic administration of STING agonists reduces local bone cancer tumor burden in a STING- and Ifnar1-dependent manner.

LLC is a murine lung adenocarcinoma cell line which has affinity to metastasize from the original injection site to pulmonary lobes and form visible tumor nodules, enabling use of this phenomenon as a measure of metastasis in our model. To test whether systemic administration of ADU-S100 (20 mg/kg i.p.) or ZA (100 μg/kg i.p.) at d3 and d7 could reduce lung metastasis, we analyzed lungs from vehicle-, ADU-S100-, or ZA-treated mice at d17 after intrafemoral LLC inoculation. We found that mice receiving ADU-S100 exhibited fewer lung tumor nodules compared to mice treated with vehicle or ZA (FIG. 6d-e of Wang, supra). Thus, systemic STING activation with ADU-S100 can inhibit both local tumor burden as well as further tumor metastasis.

Mechanistically, the antitumor effects of STING pathway activation are chiefly attributed to antigen presenting cell (APC)-mediated activation of CD8⁺ T cells. To test whether systemic STING activation can promote CD8⁺ T cell infiltration into the immunosuppressive tumor microenvironment of the bone marrow in our bone cancer model, mice were administered DMXAA (20 mg/kg i.p.) at d3 and d7 and bone marrow was collected from tumor-bearing femora 24 h after the second DMXAA injection for analysis of tumor-infiltrating lymphocytes (TILs) by flow cytometry. Importantly, we found that DMXAA treatment significantly increased the proportion of (CD11b⁻ CD3⁺) CD8⁺ T cells without significantly changing the proportion of (CD11b⁻ CD3⁺) CD4⁺ T cells in the bone marrow tumor microenvironment (FIG. 6f of Wang, supra). We further analyzed the proportion of immunosuppressive (CD3⁺ CD4⁺) Foxp3⁺, IL-17⁻ T^{reg} cells and found that DMXAA treatment decreased the proportion of T^{reg} cells in the bone marrow (FIG. 6g of Wang, supra). To test whether STING agonist-induced reduction in tumor burden is due to T cell-mediated antitumor immunity, we introduced LLC cells into the intrafemoral cavity of WT or Rag1^{-/-} mice lacking mature B and T cells, followed by vehicle or DMXAA treatment (20 mg/kg i.p. at d3 and d7 post-inoculation) and measurement of maximum thigh circumference at d17 as in FIG. 6b of Wang, supra. We found that DMXAA effectively reduced the ratio of maximum thigh circumference only in WT mice but not in Rag1^{-/-} mice (FIG. 6h of Wang, supra). Next, given that conventional type 1 dendritic cells (cDC1) have been demonstrated to be critical for cross-priming adaptive T cell responses against tumors through STING-mediated IFN-I induction, we also utilized cDC1-deficient Batf3^{-/-} mice to test whether the acute and/or long-term protective effects would depend on adaptive antitumor immunity. Upon femoral inoculation of Batf3^{+/+} or Batf3^{-/-} mice with LLC cells, only tumor-bearing Batf3^{+/+} mice but not Batf3^{-/-} mice exhibited a decrease in local tumor burden at d17 following DMXAA treatment (FIG. 6i of Wang, supra). Thus, we conclude that systemic activation of host-intrinsic STING-mediated IFN-I signaling facilitates antitumor immunity by promoting TIL entry into the normally immunosuppressive bone marrow TME.

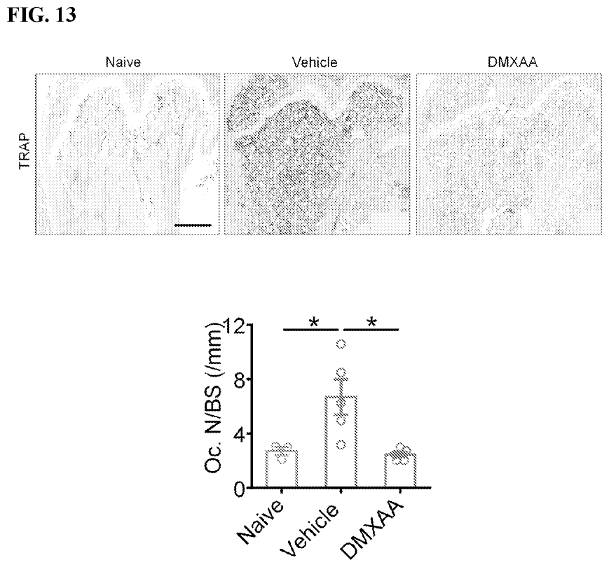

STING agonists inhibit cancer-induced osteoclast differentiation via IFN-I signaling. IFN-α and IFN-β were previously reported to inhibit the differentiation of murine and human preosteoclasts into osteoclasts. Given our data indicating that STING agonists can reduce bone destruction, we sought to determine whether the bone protective effects are mediated by direct effects on osteoclastogenesis. To this end, we measured osteoclast cell numbers in the distal tumor-bearing femora at d11 after inoculation in mice treated with vehicle or DMXAA (20 mg/kg i.p. at d3 and d7). Notably, DMXAA-treated mice exhibited far significantly fewer osteoclasts (FIG. 13), but no changes were observed in bone-forming osteoblasts (FIG. 7b of Wang, supra). To further evaluate the activity of osteoclasts and osteoblasts, we collected serum from tumor-bearing mice on BL and d17 after LLC inoculation and measured serum CTX-I and PINP levels, which are markers for bone resorption and bone formation, respectively. DMXAA could effectively reduce CTX-I levels on d17 but had no effect on serum PINP levels (FIG. 7c of Wang, supra). These data indicate that STING activation with DMXAA can suppress bone cancer-driven osteoclast formation and their bone catabolic activity.

Given that systemic STING agonist treatment reduces osteoclast numbers in vivo, we sought to determine whether STING pathway activation can promote osteoclast differentiation in vitro. Murine macrophage RAW 264.7 cells were treated with RANKL (35 ng/ml, for 6 days) to promote osteoclast differentiation in the presence of vehicle or an escalating dose of DMXAA or ADU-S100. Importantly, we found that both DMXAA and ADU-S100 dose dependently inhibited osteoclast differentiation (FIG. 7d-e of Wang, supra). Bone marrow cells from WT, STING^{gt/gt}, or Ifnar1^{-/-} mice were harvested and differentiated into macrophages with 20 ng/ml M-CSF for 3 days. These bone marrow-derived macrophages (BMDM) were further induced into osteoclasts with 20 ng/ml M-CSF and 35 ng/ml RANKL for 7 days. We collected the BMDM culture medium 24 h after incubation with DMXAA or ADU-S100 and found both agonists induced a drastic increase in IFN-α and IFN-β levels in the culture medium, although IFN-β induction was much greater (FIG. 7f of Wang, supra). Furthermore, TRAP staining showed that DMXAA or ADU-S100 treatment (30 μM each) could significantly inhibit osteoclast formation from BMDM from WT mice but not from STING^{gt/gt} or Ifnar1^{-/-} mice (FIG. 7g-h of Wang, supra). To provide further support that these effects were reliant on IFN-α/β signaling, anti-IFN-α (600 ng/ml) or anti-IFN-β (600 ng/ml) neutralizing antibodies were added to the induction medium of BMDM followed by analysis of osteoclast formation. We found that anti-IFN-β antibody could block the inhibition of osteoclast formation by DMXAA or ADU-S100 (Extended Data FIG. 5a-d of Wang, supra). Taken together, these data indicate that activation of the STING/IFN-I signaling axis can reduce osteoclastogenesis.

Our findings indicate that STING agonists produce antinociception, reduce tumor burden, and reduce bone destruction and osteoclastogenesis. One could argue that both the antinociceptive effects and the bone protective effects are secondary to T cell-mediated antitumor immunity. To test this possibility, we again introduced LLC cells into the intrafemoral cavity of WT or Rag1^{-/-} mice, followed by vehicle or DMXAA treatment (20 mg/kg i.p.) at d3 and d7 post-inoculation. Notably, bone cancer-induced mechanical and cold allodynia were reduced by DMXAA treatment in both WT and Rag1^{-/-} mice at early stages (7 d and 10 d), but not at later stages (14d; Extended Data FIG. 6a-b of Wang, supra). Likewise, DMXAA treatment led to an improvement in the bone destruction score in both WT and Rag1^{-/-} mice at d11, but not at d15 and at d17 for bone fracture (Extended Data FIG. 6c-e of Wang, supra). Thus, these data indicate that DMXAA suppresses pain and bone destruction in a T cell-independent mechanism at early stages, and thus, these effects are likely due to direct suppression of nociceptor excitability and osteoclastogenesis. As the local tumor burden increases at later stages, T cell-mediated antitumor immunity may become essential in controlling pain and bone destruction. To further verify these findings, we used Batf3^{+/+} and Batf3^{-/-} mice to establish bone cancer pain model. DMXAA treatment (20 mg/kg i.p., d3 and d7) could attenuate mechanical allodynia or cold allodynia in Batf3^{-/-} mice on d7 and d10 but not day 14 after tumor inoculation (Extended Data FIG. 7a-b of Wang, supra). DMXAA also reduced bone destruction on d8 and d11 but not d15 in Batf3^{-/-} mice (Extended Data FIG. 7c-d of Wang, supra). These data provide an additional line of evidence that DMXAA suppresses pain and bone destruction in a T cell-independent mechanism at early stages. Overall, we propose a mechanism by which STING agonists induce robust production of type-I interferons, which directly suppress nociceptor excitability and osteoclastogenesis while concurrently promoting T cell-mediated antitumor immunity. Thus, we posit that STING agonists can acutely suppress cancer pain through direct effects, while providing long term relief from bone cancer-induced pain by suppressing osteoclast-mediated bone destruction and relieving local tumor burden (FIG. 8 of Wang, supra).

### IV. EXEMPLARY EMBODIMENTS

Exemplary embodiments provided in accordance with the presently disclosed subject matter include, but are not limited to, the claims and the following embodiments:

1. A method of treating pain, the method comprising administering a therapeutically effective amount of a stimulator of interferon genes (STING) agonist to a subject in need thereof, thereby treating the pain.

2. The method of embodiment 1, wherein the STING agonist is selected from the group consisting of a cyclic dinucleotide, an amidobenzimidazole, a benzothiophene, a benzo[b]thiophene, an aza-benzothiophene, pharmaceutically acceptable salts thereof, and combinations thereof.

3. The method of embodiment 2, wherein the STING agonist is a cyclic dinucleotide or a pharmaceutically acceptable salt thereof.

4. The method of embodiment 2, wherein the cyclic dinucleotide is selected from the group consisting of 2′3′-cGAMP, 3′3′-cGAMP, cyclic diAMP, cyclic diGMP, a cyclic dinucleotide thiophosphate, pharmaceutically acceptable salts thereof, and combinations thereof.

5. The method of embodiment 4, wherein the cyclic dinucleotide thiophosphate is (2′-5′)-[P(R)]-5′-O-[(R)-hydroxymercaptophosphinyl]-P-thioadenylyl-adenosine cyclic dinucleotide (ADU-S100) or a pharmaceutically acceptable salt thereof.

6. The method of any one of embodiments 1-5, wherein the STING agonist is administered to the subject's dorsal root ganglia, skin, muscle, joint or cerebral spinal fluid (CSF).

7. The method of any one of embodiments 1-5, wherein the STING agonist is administered systemically via injection.

8. The method of any one of embodiments 1-7, further comprising administering an effective amount of an additional therapeutic agent to the subject.

9. The method of embodiment 8, wherein the additional therapeutic agent is selected from the group consisting of a steroid, a nonsteroidal anti-inflammatory drug, an opioid, a local anesthetic, PD-L1 or a derivative thereof, a PD-1 activator, a SHP-1 phosphatase activator, and combinations thereof.

10. The method of any one of embodiments 1-9, wherein the pain comprises neuropathic pain, inflammatory pain, cancer pain, or a combination thereof.

11. The method of any one of embodiments 1-10, wherein the pain is characterized by mechanical allodynia, cold allodynia, or a combination thereof.

12. The method of any one of embodiments 1-11, wherein the pain is characterized by spontaneous occurrence or ongoing occurrence.

13. A kit for the treatment of pain comprising a therapeutically effective amount of a STING agonist and instructions for use in the treatment of pain.

14. The kit according to embodiment 13, wherein the STING agonist is a compound as recited in any one of embodiments 2-5.

15. The kit according to embodiment 13 or embodiment 14, further comprising an apparatus for administering the STING agonist.

16. The kit according to any one of embodiments 13-15, further comprising at least one additional therapeutic agent.

17. The kit according to embodiment 16, wherein the additional therapeutic agent is an agent as recited in embodiment 9.

18. The according kid to embodiment 16 or embodiment 17, further comprising an apparatus for administering the additional therapeutic agent.

Although the foregoing has been described in some detail by way of illustration and example for purposes of clarity and understanding, one of skill in the art will appreciate that certain changes and modifications can be practiced within the scope of the appended claims.

In addition, each reference provided herein is incorporated by reference in its entirety to the same extent as if each reference was individually incorporated by reference.

1. A method of treating pain, the method comprising administering a therapeutically effective amount of a stimulator of interferon genes (STING) agonist to a subject in need thereof, thereby treating the pain.

2. The method of claim 1, wherein the STING agonist is selected from the group consisting of a cyclic dinucleotide, an amidobenzimidazole, a benzothiophene, a benzo[b]thiophene, an aza-benzothiophene, pharmaceutically acceptable salts thereof, and combinations thereof.

3. The method of claim 2, wherein the STING agonist is a cyclic dinucleotide or a pharmaceutically acceptable salt thereof.

4. The method of claim 2, wherein the cyclic dinucleotide is selected from the group consisting of 2′3′-cGAMP, 3′3′-cGAMP, cyclic diAMP, cyclic diGMP, a cyclic dinucleotide thiophosphate, pharmaceutically acceptable salts thereof, and combinations thereof.

5. The method of claim 4, wherein the cyclic dinucleotide thiophosphate is (2′-5′)-[P(R)]-5′-O-[(R)-hydroxymercaptophosphinyl]-P-thioadenylyl-adenosine cyclic dinucleotide (ADU-S100) or a pharmaceutically acceptable salt thereof.

6. The method of claim 1, wherein the STING agonist is administered to the subject's dorsal root ganglia, skin, muscle, joint or cerebral spinal fluid (CSF).

7. The method of claim 1, wherein the STING agonist is administered systemically via injection.

8. The method of claim 1, further comprising administering an effective amount of an additional therapeutic agent to the subject.

9. The method of claim 8, wherein the additional therapeutic agent is selected from the group consisting of a steroid, a nonsteroidal anti-inflammatory drug, an opioid, a local anesthetic, PD-L1 or a derivative thereof, a PD-1 activator, a SHP-1 phosphatase activator, and combinations thereof.

10. The method of claim 1, wherein the pain comprises neuropathic pain, inflammatory pain, cancer pain, or a combination thereof.

11. The method of claim 1, wherein the pain is characterized by mechanical allodynia, cold allodynia, or a combination thereof.

12. The method of claim 1, wherein the pain is characterized by spontaneous occurrence or ongoing occurrence.

13. A kit for the treatment of pain comprising a therapeutically effective amount of a STING agonist and instructions for use in the treatment of pain.

14. The kit according to claim 13, wherein the STING agonist is
a compound selected from the group consisting of a cyclic dinucleotide, an amidobenzimidazole, a benzothiophene, a benzo[b]thiophene, an aza-benzothiophene, pharmaceutically acceptable salts thereof, and combinations of any thereof; or
a cyclic dinucleotide selected from the group consisting of 2′3′-cGAMP, 3′3′-cGAMP, cyclic diAMP, cyclic diGMP, a cyclic dinucleotide thiophosphate, pharmaceutically acceptable salts thereof, and combinations of any thereof.

15. The kit according to claim 13, further comprising an apparatus for administering the STING agonist.

16. The kit according to claim 13, further comprising at least one additional therapeutic agent.

17. The kit according to claim 16, wherein the additional therapeutic agent is an agent as recited in claim 9.

18. The according kid to claim 16, further comprising an apparatus for administering the additional therapeutic agent.

19. The according kit to claim 13, wherein the STING agonist is a cyclic dinucleotide or a pharmaceutically acceptable salt thereof.

20. The according kit to claim 14, wherein the cyclic dinucleotide thiophosphate is (2′-5′)-[P(R)]-5′-O-[(R)-hydroxymercaptophosphinyl]-P-thioadenylyl-adenosine cyclic dinucleotide (ADU-S100) or a pharmaceutically acceptable salt thereof.
